(19) United States
(12) Patent Application Publication
(10) Pub. No.: US 2011/0070254 A1
(43) Pub. Date: Mar. 24, 2011

(54) MUTATIONS IN THE INFLUENZA A VIRUS NS1 GENE AND USE THEREOF

(75) Inventors: Earl G. Brown, Ottawa (CA); Nicole Forbes, Vanier (CA); Ping Jihui, Ottawa (CA)

(73) Assignee: UNIVERSITY OF OTTAWA, OTTAWA (CA)

(21) Appl. No.: 12/834,410

(22) Filed: Jul. 12, 2010

Related U.S. Application Data
(60) Provisional application No. 61/224,679, filed on Jul. 10, 2009.

(51) Int. Cl. A61K 39/145; C07K 14/11; C07H 21/00; A61P 31/16; C12N 15/63; C12N 5/10; C12N 7/00; C12P 21/02; C12N 7/04; C12N 7/01; A61P 37/04

(52) U.S. Cl. 424/186.1; 530/350; 536/23.72; 435/320.1; 435/325; 435/235.1; 435/69.1; 435/236

(57) ABSTRACT

NS1 variant polypeptides, proteins or functional fragments thereof are described which have useful properties for increasing viral protein synthesis, IFN induction, and IFN resistance. The NS1 variant polypeptides, proteins or functional fragments comprise a substitution in the amino acid sequence of the wild type NS1 protein at a position corresponding to Asp-2, Val-23, Leu-98, Phe-103, Ser-103, Met-106, Met-124, Asp-125, Val-180, Val-226 or Arg-227, or combinations thereof expressed alone or in infectious virus.

### FIELD OF THE INVENTION

The invention relates to influenza A viruses, and more specifically, the influenza A NS1 protein. Mutations of the NS1 protein and gene are herein described which have useful properties for increasing viral protein synthesis, virus yield, IFN induction, and IFN resistance.

### BACKGROUND OF THE INVENTION

Influenza is a very contagious respiratory infection. Influenza viruses are segmented negative-strand RNA viruses that belong to the Orthomyxoviridae family. Influenza viruses can be divided into three genera: influenza A, influenza B, and influenza C. Influenza A viruses (FLUAV) can infect a variety of species, including humans, pigs, horses, and birds, while influenza B and C viruses are generally limited to humans.

The FLUAV virus genome contains eight segments of single-stranded RNA of negative polarity, coding for two nonstructural proteins and nine structural proteins. The segmented nature of the genome allows for the exchange of entire gene segments between different viral strains during cellular co-infection.

To counteract the rapid and efficient induction of antiviral interferon (IFN) response, many viruses encode IFN antagonists. The FLUAV NS1 protein functions at multiple levels to enhance viral replication as well as to antagonize the IFN response.

The FLUAV NS1 protein is essential for virus replication in IFN-competent systems such that the NS1 deleted A/PR/8/34 mutant can only replicate in IFN unresponsive tissues or mice such as Vero cells or in STAT-1 knock-out mice (Garcia-Sastre et al., 1998). NS1 inhibits the IFN response at multiple levels including induction, synthesis and effector activities (reviewed in Garcia-Sastre & Biron, 2006; Garcia-Sastre, 2006; Krug et al., 2003; Hale et al., 2008). NS1 inhibits IFN induction through multiple inhibitory effects on transcription, such as activation of RIG-I (Mibayashi et al., 2007) and IRF3 (Talon et al., 2000) and other IFN transcription factors (Hale, Randall, Ortin, & Jackson, 2008), as well as post-transcriptional processing. In particular, host gene expression is inhibited by binding of NS1 to the 30 kD subunit of cleavage and polyadenylation specificity factor (CPSF) (Noah et al., 2003) and the poly-A binding protein nuclear I (PABPN1) {Chen, 1999 329/id} to prevent polyadenylation. NS1 also interferes with the action of IFN by directly by binding to IFN effectors such as dsRNA dependent protein kinase (PKR) (Min et al., 2007) or indirectly by binding dsRNA (Tan & Katze, 1998; Wang et al., 1999) to prevent activation of IFN effectors such as PKR and 2′5′ oligo-A synthetase that activates RNase-L (Min & Krug, 2006). In addition, NS1 acts to enhance viral protein synthesis through interactions with viral mRNA (de la Luna et al., 1995) and translation initiation factors eIF4G1 and poly-A binding protein 1 (PABP1) (Burgui et al., 2003; de la, Fortes, Beloso, & Ortin, 1995; Marion et al., 1997a). NS1 also enhances viral replication and controls apoptosis through binding and activation of the regulatory subunit of PI3K (Hale & Randall, 2007; Shin et al., 2007) and has been shown to bind influenza RNA polymerase (Marion et al., 1997b) and to function in the temporal control of transcription (Min, Li, Sen, & Krug, 2007).

Although NS1 inhibits and activates many host factors it is not clear which functions are the primary modulators for adaptive differences among FLUAVs.

Adaptation of FLUAV to a new host such as the mouse results in the selection of variants with enhanced abilities to exploit and replicate in host tissues (Brown & Bailly, 1999; Brown et al., 2001; Ward, 1997; Gabriel et al., 2005; Keleta et al., 2008). Previous mouse adaptation of the clinical human H3N2 FLUAV isolate, A/Hong Kong/1/68 (HK-wt), resulted in the selection of virulent mouse-adapted (MA) variant clones possessing specific NS1 gene mutations such as HKMA20 and HKMA20c with V23A or F103L mutations respectively (Brown, Liu, Kit, Baird, & Nesrallah, 2001).

The present inventors have used this adaptive approach in order to produce NS1 variants that have improved properties for therapeutic, diagnostic and research applications.

Influenza Vaccines

Influenza vaccines are often composed of reassortant strains composed of the six internal gene segments derived from a master donor virus (MDV) and the two segments that encode the two surface glycoproteins, hemagglutinin (HA) and neuraminidase (NA) that are derived from the corresponding antigenically relevant wild-type virus (ex. the pandemic (H1N1) 2009 wild-type virus). NS1 is coded within the six internal gene segments. The HA and NA surface proteins give rise to the immune response. Influenza vaccines are often manufactured as live attenuated vaccines or inactivated vaccines. In such live and inactivated vaccines, the virus (1) maintains the replication characteristics and phenotypic properties of the MDV; and (2) expresses the HA and NA of the virus of interest, such as the pandemic (H1N1) 2009 virus.

The MDV used to manufacture live attenuated vaccines generally has the following phenotypes conferred by its internal six gene segments: cold adapted (ca), temperature sensitive (ts), and attenuated (att). The cumulative effect of the antigenic properties and the ca, ts, and att phenotypes is that the attenuated vaccine virus replicates in the nasopharynx to induce protective immunity. The MDV used to manufacture the inactivated vaccines generally has a high yielding phenotype conferred by its internal six gene segments. The ca, ts, and att phenotypes are not desired in this case as the final vaccine does not contain any live virus.

One strategy to enhance the performance and production of influenza vaccines is to introduce genetic modifications into the MDV to produce a virus strain with improved properties. A panel of mutations in the NS1 gene is herein provided that have applications in improving influenza vaccines.

### SUMMARY OF THE INVENTION

The present invention provides new influenza NS1 variants that have properties that are desirable in the production of numerous types of vaccines as well as in research and diagnostics. Also provided herein is an influenza vaccine comprising these NS1 variants.

Accordingly, there is provided an isolated NS1 variant polypeptide, protein or functional fragment thereof comprising a substitution in the amino acid sequence of the wild type NS1 protein at a position corresponding to Asp-2, Val-23, Leu-98, Phe-103, Met-106, Met-124, Asp-125, Val-180, Val-226 or Arg-227, or combinations thereof.

In an embodiment, there is provided a NS1 variant polypeptide comprising a mutation at Asp-2, Val-23, Phe-103, Met-106, Met-106+Leu-98, Met-106+Met-124, Asp-125, Val-180, Val-226 or Arg-227 of the wild type NS1 sequence, the mutation being with any amino acid that maintains the structure and function of the NS1 protein. In non-limiting examples the amino acids may be substituted with asparagine, alanine, leucine, serine, isoleucine, isoleucine, serine, valine, valine, isolucine, glycine, alanine, isolucine and lysine residues respectively. In specific embodiments, the NS1 variant polypeptides may include one or more of the following mutations: D2N, V226I, V23A, F103L, F103S, M106I, M106I+L98S, M106V, M106V+M124I, D125G, V180A and R227K.

In specific yet non-limiting embodiments, the NS1 variant polypeptide comprises the amino acid sequence of the NS1 protein having a mutation from one of the following: D2N (SEQ ID NO:1), V226I (SEQ ID NO:2), V23A (SEQ ID NO:3), F103L (SEQ ID NO:4), F103S (SEQ ID NO:5), M106I (SEQ ID NO:6), M106I+L98S (SEQ ID NO:7), M106V (SEQ ID NO:8), M106V+M124I (SEQ ID NO:9), D125G (SEQ ID NO:10), V180A (SEQ ID NO:11) and R227K (SEQ ID NO:12).

Polypeptides as described herein will preferably involve purified or isolated polypeptide preparations. In certain embodiments, purification of the polypeptide may utilize recombinant expression methods well known in the art, and may involve the incorporation of an affinity tag into the expression construct to allow for affinity purification of the target polypeptide.

Fragments of the above polypeptides are also included herein, but are not limited to amino acid sequences wherein one or more amino acids are deleted. For example, but not to be considered limiting, a fragment may exist when one or more amino acids from the amino terminal, carboxy terminal or both are removed. Further, one or more amino acids internal to the polypeptide may be deleted.

It is also contemplated that the above polypeptides may comprise one or more amino acid substitutions, additions, insertions, or a combination thereof in the sequences shown herein. Preferably, the amino acid sequence exhibits greater than about 90% homology, more preferably greater than about 91%, 92%, 93%, 94%, 95%, 96%, 97%, 98%, 99% or about 100% homology to the sequence(s) described herein. The degree of homology may also be represented by a range defined by any two of the values listed above or any value therein between.

It is further contemplated that the amino acid sequence comprises greater than about 70%, more preferably about 80%, 85%, 90%, 91%, 92%, 93%, 94%, 95%, 96%, 97%, 98%, 99% or about 100% identity with the amino acid sequence(s) described herein. Further, the degree of identity may be represented by a range defined by any two of the values listed or any value therein between. Methods for determining % identity or % homology are known in the art and any suitable method may be employed for this purpose.

There is further provided an isolated polynucleotide which encodes the above-described NS1 variant polypeptide, protein or functional fragment thereof. Embodiments of such polynucleotides can be derived from the wild-type nucleotide sequence shown in FIG. 1b, or may comprise one of the mutant sequences D2N (SEQ ID NO:13), V226I, (SEQ ID NO:14), V23A (SEQ ID NO:15), F103L (SEQ ID NO:16), F103S (SEQ ID NO:17), M106I (SEQ ID NO:18), M106I+L98S (SEQ ID NO:19), M106V (SEQ ID NO:20), M106V+M124I (SEQ ID NO:21), D125G (SEQ ID NO:22), V180A (SEQ ID NO:23) or R227K (SEQ ID NO:24) also shown therein, including minor variations thereof.

In addition to DNA sequences, the cDNA and RNA transcripts of the above described polynucleotides are also provided. For example, the RNA sequences can be rescued into influenza genomes to create influenza master donor virus strains containing the desired mutations using reverse genetics.

The nucleotide sequences provided by the present invention may be part of a larger nucleotide sequence or nucleotide construct optionally comprising one or more regulatory sequences, for example promoters, terminators and the like. By the terms “regulatory sequence”, “regulatory region”, “regulatory element” it is meant a portion of nucleic acid typically, but not always, upstream of the protein or polypeptide coding region of a nucleotide sequence, which may be comprised of either DNA or RNA, or both DNA and RNA. When a regulatory region is active, and in operative association with a nucleotide sequence of interest, this may result in expression of the nucleotide sequence of interest. A regulatory element may be capable of mediating organ specificity, or controlling developmental or temporal nucleotide sequence activation. A “regulatory region” includes promoter elements, core promoter elements exhibiting a basal promoter activity, elements that are inducible in response to a stimulus, elements that mediate promoter activity such as negative regulatory elements or transcriptional enhancers. “Regulatory region”, as used herein, also includes elements that are active following transcription, for example, regulatory elements that modulate nucleotide sequence expression such as translational and transcriptional enhancers, translational and transcriptional repressors, upstream activating sequences, and mRNA instability determinants. Several of these latter elements may be located proximal to the coding region.

In further embodiments there are provided vectors comprising the above-described polynucleotides. The vector may be a plasmid, a cosmid, a phage, a virus, or a fragment of a virus. The vector can be an expression vector.

There is further provided a cell comprising a polynucleotide or vectors as described above, as well as a recombinant influenza virus comprising a polypeptide or polynucleotide possessing the mutations as described herein. Compositions containing the recombinant influenza viruses are also provided.

Also provided herein is an NS1 mutant influenza virus or an influenza viral master donor virus which incorporates the coding sequence of a NS1 variant polypeptide, protein or functional fragment as described above, which when incorporated into the influenza viral master donor virus, causes the virus to exhibit an increased IFN inducing and high protein synthesis phenotype.

Without wishing to be limiting in any manner, an NS1 mutant influenza virus as described herein may include viruses possessing NS1 genes of defined sequence, including viruses made by reassortment, or recombinant means including genetic engineering. Recombinant NS1 mutant influenza viruses include reassortant influenza viruses generated by using genetic engineering.

In addition, there is provided an immunogenic composition containing an effective amount of the NS1 mutant influenza virus described above. In certain embodiments, the influenza virus in the immunogenic composition is formulated for administration orally, intradermally, intramuscularly, intraperitoneally, intravenously, or subcutaneously.

The above NS1 variant polypeptides and polynucleotides can be used in a method to increase influenza protein and/or virus production, for example by administering a virus or composition as described herein to a cell, cell extract, tissue and/or tissue extract, and inducing protein and/or virus production in said cell, cell extract, tissue and/or tissue extract Without wishing to be limiting in any manner, the cell, cell extract, tissue and/or tissue extract may comprise or be derived from fertilized chicken eggs, mammalian kidney cells or other cell, cell extract, tissue and/or tissue extract used for growing influenza virus or producing protein. Such proteins can be used for vaccination or other therapeutic applications.

The above NS1 variant polypeptides and polynucleotides can also be used in a method to increase the safety and immune response to recombinant influenza vaccines, for instance by administering an NS1 variant polypeptide, protein or functional fragment thereof as described herein, a polynucleotide encoding the polypeptide, protein or functional fragment or a composition comprising the polypeptide, protein or functional fragment or the polynucleotide, in an amount sufficient to increase IFN production.

The above NS1 variant polypeptides and polynucleotides can also be used in a method to increase the tumor specificity of oncolytic influenza viruses, for instance by administering a composition as described herein in an amount sufficient to increase said tumor specificity.

Also provided herein is a method of inducing an immune response in a subject, comprising administering an NS1 variant polypeptide, protein or functional fragment thereof as described herein, a polynucleotide encoding the polypeptide, protein or functional fragment or a composition comprising the polypeptide, protein or functional fragment or the polynucleotide, to the subject in an effective amount to induce said immune response.

In addition, there is provided a method of inducing an immune response in a subject, comprising administering an NS1 mutant influenza virus to said subject in an effective amount to induce said immune response. In other non-limiting embodiments the influenza virus can be provided in a formulation for administration to the subject for instance by administration orally, intradermally, intramuscularly, intraperitoneally, intravenously, or subcutaneously.

Additionally, there is provided herein a method of producing an influenza vaccine comprising: (a) growing a virus as described above; (b) purifying the virus; and (c) combining the purified virus with a pharmaceutically acceptable excipient. In a non-limiting embodiment, the virus may be grown in embryonated chicken eggs or mammalian kidney cells. In addition, yet without wishing to be limiting in any manner, it is envisioned that the virus may be inactivated prior to step (c). Other steps and variations may also be encompassed within the aforementioned method, based on common vaccine production techniques known in the art.

Additionally, there is provided herein a method of producing an influenza vaccine comprising: (a) preparing an NS1 variant polypeptide, protein or functional fragment thereof as described above or polynucleotide encoding the NS1 variant polypeptide, protein or functional fragment thereof; (b) purifying the polypeptiptide or polynucleotide; and (c) combining the NS1 variant polypeptide, protein or functional fragment thereof or said polynucleotide with an influenza antigen and a pharmaceutically acceptable carrier or excipient. In a non-limiting embodiment, the polypeptide or polynucleotide may prepared using cell-based or synthetic methods.

A subject in the method(s) described herein may be a mammalian subject, for example, but not limited to mouse, cow, sheep, goat, pig, dog, cat, rat, rabbit, primate, or human. In an embodiment, which is not meant to be limiting, the subject is a human.

Those skilled in the art will recognize, or be able to ascertain using no more than routine experimentation, numerous equivalents to the specific products and procedures described herein. Such equivalents are considered to be within the scope of this invention and are covered by the following claims.

### BRIEF DESCRIPTION OF DRAWINGS

Embodiments of the present invention will now be described, by way of examples only, with references to the attached Figures, wherein:

FIG. E1a shows an alignment of mouse adapted NS1 mutant protein sequences, together with the wild type NS1 protein sequence.

FIG. E1b shows the nucleic acid sequences of the genes encoding the wild type and mouse adapted mutant NS1 proteins.

FIG. 1 shows a map of mouse adapted NS1 mutations and replication of recombinant WSN viruses expressing NS1 mutants in MDCK cells. In 1a, NS1 mutations are indicated with arrows on a genetic map of NS1 interaction sites with binding factors indicated. In 1b, the replication in MDCK cells of rWSN wt and recombinant viruses possessing NS1 HK-wt and mutant NS1 variants as indicated. Cells were infected at a moi of 0.02 in triplicate and monitored for infectious yield over 72 hrs; data are shown plus or minus 1 standard deviation; * indicates P<0.05.

FIG. 2 shows the effect of NS1 mutations on protein synthesis in canine, human and mouse cells. In 2a, monolayers of MDCK cells were infected at moi=2 with rWSN viruses possessing the HK-wt-NS1 gene or defined NS1 mutants for 8 h before sample collection for western blotting using rabbit anti-NS1 serum, anti-WSN virus serum for HA, NP, and M1 proteins, and anti-actin for loading control. Input virus and the NP protein detectable at 2 hr are shown to indicate standardization of infection. In 2b, infection of monolayers of human A549 cells were performed as described for 2a. In 2c, infection of mouse M1 cells performed as described for 2a. FIG. 2d-f show graphs of relative levels of accumulated protein detected by densitometry for infections in 1a-c that were normalized relative to HK-wt and actin; the levels of NS1 proteins represent the average and standard error of duplicate experiments of NS1 protein synthesis in MDCK and M1 cells. Significant differences relative to HK-wt levels are indicated with an asterisk (p<0.05 by t test).

FIG. 3 shows the effect of NS1 mutations on IFNβ induction in mouse M1 cells. In 3a M1 cells were infected with rWSN, HK-wt or rWSN recombinant viruses possessing HK-wt or mutant HK NS1 genes at a moi=2 with assay of IFNβ assay by ELISA, (n=2-3) at 24 h post-infection (pi); values indicate averages plus or minus standard errors or deviations; * indicates p<0.05 by t test. FIG. 3b shows the IFNβ assay of M1 cells infected with mouse adapted variants possessing NS1 mutants shown in 3a, and Table 1.

FIG. 4 shows that NS1 mutations confer increased protein synthesis following IFNβ pretreatment. FIG. 4a shows M1 cells that were untreated or pretreated with 200 U/ml of IFNβ for 6 hrs before infection and assay of NS1 protein synthesis as described in FIG. 2a. Treated and untreated samples of HK-wt NS1 proteins are shown after analyses in the same gel for comparison. FIG. 4b is a graphical presentation of NS1 protein synthesis following IFN pretreatment, normalized to HK-wt and actin. FIG. 4c is a side-by-side comparison of IFN treated and untreated infections with wild type and NS1 mutant samples that are presented in FIG. 4a illustrating the IFN-resistant protein synthesis of the NS1 protein in NS-mutant-infected cells. FIG. 4d shows the ratio of NS1 protein synthesized with and without IFNβ pretreatment from densitometry analysis of the data in FIG. 4c. (untreated/IFN pretreatment).

FIG. 5 shows the infectious yields of NS1 mutants in untreated and IFNβ pretreated M1 cells. The infectious yield of viruses in infected M1 cells is shown without pretreatment (5a) and with 200 U/ml of mouse IFNβ pre-treatment (5b). FIG. 5c shows the ratio of yield with and without IFNβ is indicated as (untreated/IFN pretreatment). Values are the averages with standard deviation of n=3-5 experiments; significant differences relative to HK-wt are indicated with an asterisk (P<0.05 by t test).

FIG. 6 depicts the coimmunoprecipitation of HK-wt and all mutant NS1 proteins with CPSF30-F2/F3. In FIG. 6a, recombinant NS1 proteins (2.0 μg) were mixed with FLAG-tagged CPSF30-F2/F3 before blotting in parallel using anti-NS1 or anti-FLAG monoclonal antibody respectively to demonstrate the input. Pull down samples were blotted in side-by-side comparisons for immunoglobulin and NS1 protein to demonstrate association of NS1 with CPSF30-F2/F3. FIGS. 6b and 6c show CPSF-F2F3 association curves that were generated by measuring the association relative to varying amounts of NS1 protein. Densitometry data was calculated from non-saturated exposures of 6b, corrected for differences in the input NS1 loading controls, and used to plot association curves (6c). Western blots show input NS1 and CPSF30-F2/F3 loading controls (LC), as well as α-Flag pulldown controls with each NS1 protein.

FIG. 7 shows the binding of HK-wt and all mutant NS1 proteins with PABP1. In 7a, glutathione Sepharose pulldown of HK-wt and all HK-NS1 mutant proteins by PABP1-GST is shown as a side-by-side comparison of the 1.0 μg input NS1 with 3 μg of PABP1-GST. Proteins were detected using anti-NS1 or anti-GST antibodies for western blot. FIGS. 7b and 7c show PABP1 binding curves that were generated by measuring binding relative to varying amounts of NS1 protein. Densitometry data was taken from non-saturated exposures of the pulldowns and adjusted for differences in the input NS1 loading controls as well as for the background association of His-NS1 with the glutathione Sepharose resin. Duplicate analysis of binding using 2 ug of NS1 were used to test statistical significance that is shown with an asterisk for P<0.05 by t test. Controls for loading “LC” (A) and nonspecific binding to GST beads are shown (GST). FIG. 7c is a model of enhanced translation through increased NS1 interaction with the translation initiation complex and RNA as well as other possible roles involving dsRNA and PKR binding. FIG. 7d shows the adaptive mutations (shown in space filling models) that define a band on the side of the NS1 dimer (in blue and green) composed of 2 separately crystallized domains. An arrow points to the expected central location of amino acid 23 relative to the effector domain in the intact molecule. FIG. 7e depicts a proposed model of NS1 enhancement of viral protein synthesis via interactions with translation initiation factors eIF4GI, PABPI, with viral mRNA, and via effects on the IFN antagonistic response.

FIG. 8 shows that mouse adapted NS1 mutations enhance viral growth in vitro in mouse kidney epithelial (M1) cells irrespective of IFN pretreatment. M1 cells were left untreated (a, b) or pretreated with 1000 U/mL murine IFNβ for 24 hours (c, d). Cells were then infected at a MOI of 0.02 with recombinant A/Hong Kong/1/1968 viruses expressing wild-type Hong Kong NS1 or mutant NS1 genes as indicated. Following infection, the cells were supplemented with Serum-Free MEM in the presence of 0.5 μg/mL trypsin. Supernatant samples were taken at 12, 24, 48 and 72 hours post infection, and assayed for viral yield by plaque assay in MDCK cells in triplicate. Values are shown as means plus and minus standard deviation.

FIG. 9 shows that mouse adapted NS1 mutations enhance virulence in the mouse. CD-1 mice were intranasally infected with a 5×10⁶ dose of recombinant A/Hong Kong/1/1968 viruses expressing wild-type Hong Kong NS1 or mutant NS1 genes (5 mice per virus treatment group). Mice were monitored for progression of disease for 14 days following infection. (a) percent survival; (b) average percent body weight. Values are shown as the means plus and minus the standard deviation.

FIG. 10 shows that adapted NS1 mutations increase average antibody levels relative to wild type in mice using two different influenza strains (WSN and PR8). The exception was for the WSN F103L mutant which did not. These 2 virus strains were chosen because they differed in level of growth in the mouse with the PR8HK-NS1 virus growing to higher levels than WSN. This allowed us to assess the effects of the F103L and M106I mutation in viruses with different growth properties (high and low). We showed that both these mutations enhanced seroconversion of the PR8 virus but only the M106I mutation increased seroconversion in the WSN virus infected mice. Mice were infected with the same dosages of live viruses (groups of mice each were infected with 1×10⁵ pfu given intranasally), with blood collection 21 days after infection. Sera were treated with Vibrio cholera neuraminidase ( 1/10 volume of 100 units per ml) overnight at 37° C. and then heat treated at 56° C. for 0.5 hr to inactivate inhibitors before HAI assay using 8 HA units of WSN or PR8 viruses using serial 2 fold dilution of serum beginning with 1/20 dilution. Immune responses for WSN H1N1 that possessed the HK NS1 genes were tested for viruses possessing HK-wild type (WSN HKNS wt), HK NS1 F103L mutant, and HKNS1 M106I mutant resulted in average HAI titres of 27, 13 and 60 respectively (values <20 were recorded as 0). These viruses therefore induced a very low immune response, although average antibody titers measured by HAI were higher for the HKM106I mutant. Immune responses were also tested for these mutations in the PR8 virus backbone for viruses possessing HK-wild type (PR8HKNS wt), HK F103L mutant (PR8HKNS F103L), and HK F103L+M106I mutant (PR8HKNS F103L+M106I) to yield HAI titres of 320, 530 and 747 respectively. Both the HK F103L mutant and HK F103L+M106I mutant therefore resulted in increased average antibody levels.

FIG. 11 shows the effect of NS1 mutations on RNA polymerase activity normalized to A/HK/1/68 wild-type activity. Polymerase activity was measured by comparison of renilia luciferase activity from a plasmid driven with a CMV promoter relative to a firefly luciferase construct that possessed the 5′ and 3′ ends of NP genome segment. The control assay contains polymerase components only: PB1, PB2 PA and NP and the reference wild type NS1 sample has NS1-HK-wt added for reference to the HK-mouse adapted mutant NS1 plasmids. All samples were normalized to the control (HK(3P+NP)). The wild type segment 8 has an inhibitory effect on RNA polymerase activity that it reversed by the F103L, D2N, M106I, M106V, M124I, D125G, R227K, L98S, V180A, but was not increased by the V23A or V226I. Values are the average of 2 independent technical assays that were each performed in triplicate. Raw data from each technical replicate is shown in FIG. 12.

FIG. 12 shows the Luciferase output values from each technical replicate is depicted in FIG. 11.

### DETAILED DESCRIPTION

Described herein is a panel of adaptive NS1 gene mutations that are multifunctional, affecting protein synthesis, IFN induction, and IFN resistance.

These mutations result in the exhibition of properties desirable for influenza vaccines, The two major properties of (1) increased interferon induction, and (2) increased viral protein synthesis and virus yield have been observed in one or more of mouse, canine, and human cell lines. Accordingly, the introduction of one or more of these mutations in the influenza viruses or MDV used to produce influenza vaccine can be used to (1) enhance the manufacturing yield of viral antigen, and (2) enhance the immune response in live attenuated vaccines.

The gene mutations were produced by NS1 adaptation to high virulence in a novel host, i.e. a mouse model, and systematic analysis of the adaptive roles of the NS1 mutations on FLUAV gene expression.

A total of 12 mouse-adapted NS1 mutants—D2N, V23A, F103L, F103S, M106I, M106I+L98S, M106V, M106V+M124I, D125G, V180A, V226I and R227K—were derived from prototype human A/Hong Kong/1/68 (HK-wt) H3N2. The majority of the mutations resided within binding sites for host translation and transcription factors. An alignment of the mutant protein sequences together with the HK-wt sequence is seen in FIG. E1a.

In general, adaptation of the NS1 gene was associated with multifunctional mutations that increased protein synthesis and that affected both IFN induction and IFN resistance (see Table 1 for summary).

TABLE 1
Summary of multifunctional mutations

| | | | | IFN |
|---|---|---|---|---|
| | Increased | | | resistant |
| | Protein | Increased | Induction of | Protein |
| Mutation | Synthesis | Yield | IFN | synthesis |
| D2N | x | x | NT | x |
| V23A | x | x | x | x |
| F103L | x | x | x | x |
| F103S | NT | x | NT | NT |
| M106I | x | x | x | x |
| M106I + L98S | x | x | x | x |
| M106V | x | X | x | x |
| M106V + M124I | NT | X | NT | NT |
| D125G | X | X | NT | X |
| V180A | x | x | x | x |
| V226I | x | x | x | |
| R227K | X | X | NT | X |

NT—not tested

Increased protein synthesis: With the exception of the HK-NS1 V23A mutant that could not be rescued onto the A/WSN/33 backbone, all other mutants demonstrated an increased ability to synthesize NS1 protein in mouse, human and/or canine cells. Increased protein synthesis was associated with increased yield for all NS1 mutants except for F103L and M106V.

Testing of protein synthesis in M1 cells with HK viruses possessing NS1 mutations has shown increased protein synthesis for all mutations except V226I (not tested, F103S, M106V+M121I).

IFN induction: The lack of uniform association of gene expression with yield was shown to be due in part to increased induction of interferon, which was seen for all NS1 mutants.

IFN resistance: When assaying for the effects of IFN pretreatment all mutants conferred increased IFN-resistant protein synthesis and in addition with IFN-resistant replication seen for all except V226I and untested mutants (F103S and M106V+M124I)

These mutations can be used to increase influenza protein production for the purpose of producing proteins for vaccination or other therapeutic applications. In addition, the increased IFN production associated with these mutations can be used to increase the safety and immune response to recombinant influenza vaccines. It is also envisioned that these mutations can be used to increase the tumor specificity of oncolytic influenza viruses.

Accordingly, there is provided herein isolated NS1 variant polypeptides, proteins or functional fragments thereof which comprise a substitution in the amino acid sequence of the wild type NS1 protein at a position corresponding to Asp-2, Val-23, Leu-98, Phe-103, Met-106, Met-124, Asp-125, Val-180, Val-226, or Arg-227, or combinations thereof. Particular NS1 variant polypeptides include mutations at Asp-2, Val-23, Phe-103, Met-106, Met-106+Leu-98, Met-106+Met-124, Asp-125, Val-180, Val-226, or Arg-227 of the wild type NS1 sequence. The mutations may be with any amino acid that maintains the structure and function of the NS1 protein, for instance: alanine, leucine, isoleucine, isoleucine, serine, valine or isoleucine residues. In specific embodiments, the NS1 variant polypeptides include one or more of the following mutations: D2N, V226I, V23A, F103L, F103S, M106I, M106I+L98S, M106V, M106V+M124I, D125G, V180A and R227K.

There is also provided polynucleotides which encode the above-described NS1 variant polypeptides, proteins or functional fragments thereof. Such polynucleotides can be derived from the wild-type nucleotide sequence shown in FIG. E1b, or may comprise one of the mutant sequences D2N, V226I, V23A, F103L, F103S, M106I, M106I+L98S, M106V, M106V+M124I, D125G, V180A or R227K also shown therein, including minor variations thereof.

The term “minor variations thereof” is intended to include changes in the nucleotide sequence which do not affect its essential nature, for example minor substitutions of nucleotides for one another. Conservative changes in the nucleotide sequence which give rise to the same protein or polypeptide will clearly be included, as will changes which cause conservative alterations in the amino acid sequence which do not affect adversely the properties of the protein or polypeptide.

Each of these nucleotide sequences may be associated with further elements such as suitable stop and start signals and other 5′ and 3′ non-coding sequences, including promoters, enabling expression of the sequence. Such further elements may be those associated with the sequence in its naturally-occurring state or may be heterologous to that sequence. Generally speaking, if a nucleotide homology of at least 75% is present and there are no out-of-frame changes to the sequence, the sequence is regarded as a “minor variation”. Preferably, the sequence is at least 80, 85, 90, 95 or 99% homologous.

The above sequences may be expressed in any suitable host. Accordingly, there is also provided herein vectors comprising the above-described polynucleotides, wherein the vector may be a plasmid, a cosmid, a phage, a virus, or a fragment of a virus. The vector can be an expression vector. There is further provided a cell comprising the above polynucleotides or vectors, as well as a recombinant influenza virus comprising the polypeptides or polynucleotides herein described. Compositions containing the recombinant influenza viruses are also provided.

It is envisioned that one or more of the adaptive NS1 gene mutations described herein can be incorporated into a reassortant virus having, for instance, one or more of the following non-limiting phenotypes: temperature-sensitive, cold-adapted, attenuated.

Immunogenic compositions may also be prepared comprising an immunologically effective amount of the recombinant and or reassortant influenza viruses as described above.

A live attenuated influenza vaccine may also be prepared which incorporates one or more of the adaptive NS1 gene mutations described herein, or by preparing the described viruses in a composition comprising, or for administration with, suitable adjuvants, excipients or acceptable pharmaceutical carriers.

Similarly, a split virus or killed virus vaccine may also be prepared which incorporates one or more of the adaptive NS1 gene mutations described herein, or by preparing the described viruses in a composition comprising, or for administration with, suitable adjuvants, excipients or acceptable pharmaceutical carriers.

A method of stimulating the immune system of an individual to produce a protective immune response against influenza virus is also provided. The method comprises administering to the individual an immunologically effective amount of the above-described recombinant influenza virus in a physiologically acceptable carrier, or one of the above-described compositions.

The term “effective amount” means that amount of a drug or pharmaceutical agent that will elicit the biological or medical response of a tissue, system, animal, or human that is being sought, for instance, by a researcher or clinician. Furthermore, the term “immunologically effective amount” means any amount which, as compared to a corresponding subject who has not received such amount, results in improved immune response, or a decrease in the rate of advancement of a disease or disorder. The term also includes within its scope amounts effective to enhance normal physiological function.

Other chemistry terms herein are used according to conventional usage in the art, as exemplified by The McGraw-Hill Dictionary of Chemical Terms (1985) and The Condensed Chemical Dictionary (1981).

### EXAMPLES

### Materials and Methods

Viruses and cells. The prototype A/HK/1/68-wt (HK-wt) virus H3N2 human clinical isolate was used to derive sister clones by MDCK plaque isolation. Virus stocks were prepared in MDCK or specific pathogen free eggs (Animal Diseases Research Institute, Ottawa) and infectivity was measured by MDCK cell plaque assay (Brown, 1990). Mouse M1 cells (kidney epithelium) and human A549 cells (lung epithelium) were purchased from ATCC.

Plaques assay. Virus samples were diluted in PBS before application to PBS washed (2×) monolayer cultures of MDCK cells, in 6 well 35 mm plates, with adsorption at 37° C. for 30 minutes before overlay with 3 mL of MEM with Earle's salts (Invitrogen, Burlington) supplanted to contain 0.65% agarose and 1.0 ug/mL trypsin. Infected plates were incubated at 37° C. for 3 days before fixation with an equal volume of Carnoy's fixative (75% methanol, 25% acetic acid) for 30 minutes. Overlays were removed in a stream of water and monolayers were stained with 0.1% crystal violet in water for 0.5 hr. before drying and counting plaques. Virus titers were calculated per mL, were determined by multiplying the plaque count times the dilution factor.

Hemagglutination Inhibition (HAI) Assay. Adult, 6-8 week old female CD-1 mice (Charles River, Quebec) were infected with 1×10⁵ pfu of test viruses given intranasally, with blood collection 21 days after infection. Blood was clotted at 37° C. for 1 hr followed by 4° C. incubation overnight before collecting the supernatant serum. Sera were treated with Vibrio cholera neuraminidase ( 1/10 volume of 100 units per ml) overnight at 37° C. and then heat treated at 56° C. for 0.5 hr to inactivate inhibitors before HAI assay using 8 HA units of WSN or PR8 viruses. Sera were treated with Vibrio Cholera neuraminidase and 56° C. to inactivate inhibitors before use. Immune and non-immune rabbit were specific for WSN and PR8 viruses were sued as positive and negative controls respectively. Sera were first subjected to serial 2 fold dilutions in PBS beginning with 1/10 dilution in a 96 well v-bottom plate and then 25 μL volumes were mixed with equal volumes of PBS containing 8 HA units of WSN or PR8 viruses as appropriate. Virus serum mixtures were incubated at 37° C. for 1 hr before mixing with 50 μL of a 1% suspension of chicken red blood cells (Canadian Food Inspection Agency, Ottawa) and incubation at 21° C. for 45 minutes. Red blood, and positive and negative serum controls were included with each assay. HAI titers were determined as the reciprocal of dilution that inhibited agglutination as confirmed by “tear-drop” testing of red blood cell pellets (tilting to vertical of assay plate for 30 seconds to confirm inhibition of agglutination as evident by red blood cell flow under gravity). The limit of detection of HAI tires was 1/20 dilution+20; values <20 were recorded as 0.

Directed evolution. Seven clonally derived stocks of the human prototype clinical isolate, A/HK/1/68 (H3N2) (designated as HK-(sister clone#)) (see Table 1), were serially passaged for 21 cycles of infection in mouse lung (MA21 viruses) before isolation of mouse adapted clones obtained by 2 serial plaque isolations on MDCK cells as described previously (Brown, Liu, Kit, Baird, & Nesrallah, 2001; Keleta, Ibricevic, Bovin, Brody, & Brown, 2008). The NS1 and NS2 genes of all HK sister clone stocks were confirmed to be identical to the wild-type sequence of parental HK virus. The mouse adapted variants were named as MA (sister clone#)(isolate#) (ie MA41). NS1 gene sequences were determined by direct sequencing of RT-PCR amplified cDNA as previously described (Brown, Liu, Kit, Baird, & Nesrallah, 2001).

Reverse genetics. Genome segment 8 of A/HK/1/68 and each A/HK/1/68-MA mutant were inserted into the pHH21 plasmid directly from cDNA using recombinational cloning (Wang et al., 2008) and were rescued into the WSN virus backbone as described by Kawaoka (Neumann et al., 1999). Successful virus rescue was assessed by the detection of cytopathology during 2 passages in MDCK cells as well as plaque assay and hemagglutination assay following an additional egg passage. Viral NS1 gene sequences were confirmed for rescued viruses.

Interferon Sensitivity Assay. The effect of interferon treatment on viral replication was determined by plaque assay of supernatants from cell monolayers that had been pretreated with 200 IU of IFNβ (Sigma-Aldrich, Canada; #I9032) for 24 h before infection for 0.5 h, at a moi of 2 pfu. Cells in 6 well dishes (1.5×10⁶ cell/dish) were washed 2 times with PBS before infection by incubation with 3×10⁶ pfu for moi=2, in 0.2 ml volumes followed by washing, twice with 3 ml of PBS, and further incubation at 37° C. for 24 hr in the presence of serum free minimal essential medium supplemented with 1 μg/ml trypsin. Input virus was not detectable after 24 hr incubation (<10 pfu/ml) in control experiments where culture supernatants were removed at t=0 h and incubated in parallel for 24 hour. IFN sensitivity was measured as the ratio (yield with IFN pretreatment/yield untreated).

Interferon Induction Assay. For IFN induction M1 cells were infected at moi=2 and incubated in serum free medium without trypsin for 24 h. Mouse IFNβ was titrated relative to IFN standards by commercial ELISA as described by the manufacturer, PBL Biomedical Laboratories (New Jersey, USA).

Protein Gel Electrophoresis and Western blot. Infected cells were fractionated by SDS PAGE using 12.5% acrylamide gels as described previously (Brown, 1990). Western blots employed rabbit antiserum raised against purified A/WS/33 or recombinant A/HK/1/68 NS1 protein detected with alkaline phosphatase labeled goat-anti-rabbit secondary antibody as described previously (Hu et al., 2005) or HRP conjugated goat-anti-rabbit (Sigma Chemical, Burlington) and SuperSignal West Pico chemiluminescent substrate (Pierce). Quantification employed densitometry using the UN-SCAN-IT Gel version 6.1 software (Silk Scientific Corp).

CPSF binding. Recombinant NS1 proteins with amino terminal 6×His tags were synthesized as described previously (Hu, Rocheleau, Larke, Chui, Lee, Ma, Liu, Omlin, Pelchat, & Brown, 2005) in BL21 pLysS E. coli using pET17b plasmids for 16 h at 21° C. with 10 μM of IPTG except that the soluble fraction was employed for purification and was dialyzed against PBS. Purified NS1 protein was quantified using the Bio-Rad Protein Assay and standardized by comparative western blot. Plasmids were constructed by insertion of the NS1 genes of HK-wt and each mutant produced by PCR mutagenesis into pET17b after PCR amplification using pfu Turbo polymerase (Stratagene, La Jolla, Calif.). CPSF30 or the CPSF30cF2F3 fragment was expressed in 1.5×10⁷ 293T cells transfected with 30 μg of pCAGGS-CPSF30-Flag or pCAGGS-CPSF30-F2/F3-Flag plasmid (obtained from L. Martinez-Sobrido, Mt. Sinai school of Medicine) in 112 μl of Lipofectamine 2000 transfection reagent (Invitrogen, Burlington, Ont.) for 24 hrs before lysis with 100 mM Tris, 250 mM NaCl, 0.5% NP-40, and 0.5% DOC, pH 8.5. Pull down experiments employed the lysate from 5×10⁵ 293T cells, 1 ug of anti-FLAG M1 monoclonal antibody (Sigma-Aldrich, Canada), defined amounts of NS1 protein, and 20 μl of protein G Dyna-beads (Invitrogen, Burlington, Ont,) in a 0.25 ml volume with rotation for 2 hr at room temperature. Beads were washed three times in lysis buffer for 10 minutes before Western blotting.

NS1-PABP1 Pulldown. PABP1-GST and GST proteins were expressed from pGEX2T-PABP1 or pGEX4T vectors (from Juan Ortin, Madrid) respectively by IPTG induction in BL21(DE3) pLysS E. coli and then column purified using Glutathione Sepharose 4B Resin (Amersham Biosciences) according to the manufacturer. Purified protein was dialyzed against PBS at pH 7.4 and quantified by Bio-Rad Protein Assay (BioRad Laboratories).

Defined amounts of NS1 and 3 μg of PABP1-GST or 1 μg of GST (negative control) were incubated together in binding buffer 150 mM NaCl, 5 mM sodium phosphate, 1% Triton X-100, 2 mM EDTA at pH 8.5 supplemented to 10% BSA for 2 hours at room temperature in the presence of 10 μL of Glutathione Sepharose 4B Resin (Amersham Biosciences). The resin was then washed 3 to 5 times with Burgui Binding Buffer before Western blot. Control binding reactions that possessed GST bound to beads demonstrated nonspecific binding to GST for the 23A and 106I+98S and 106V mutants thus necessitating the subtraction of background values from the NS1 binding values for these samples. Nonspecific binding of NS1 mutants to GST+GSR-beads was not reduced by further washing, addition of 0.5% deoxycholate or higher salt (not shown).

Statistical analysis. Replicate assays (n=2-5) were titrated in duplicate and values given as means with standard deviations or standard errors (for n=2). Significance was measured using the student's t test.

### Example 1

### Derivation of Mouse-Adapted NS1 Mutants

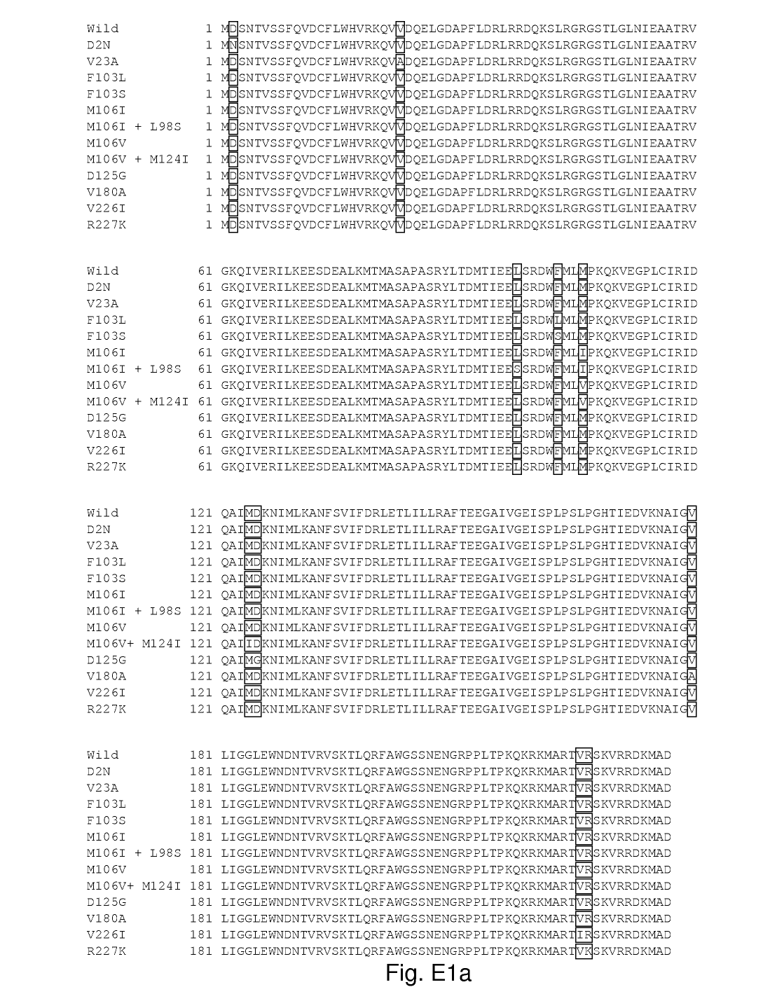

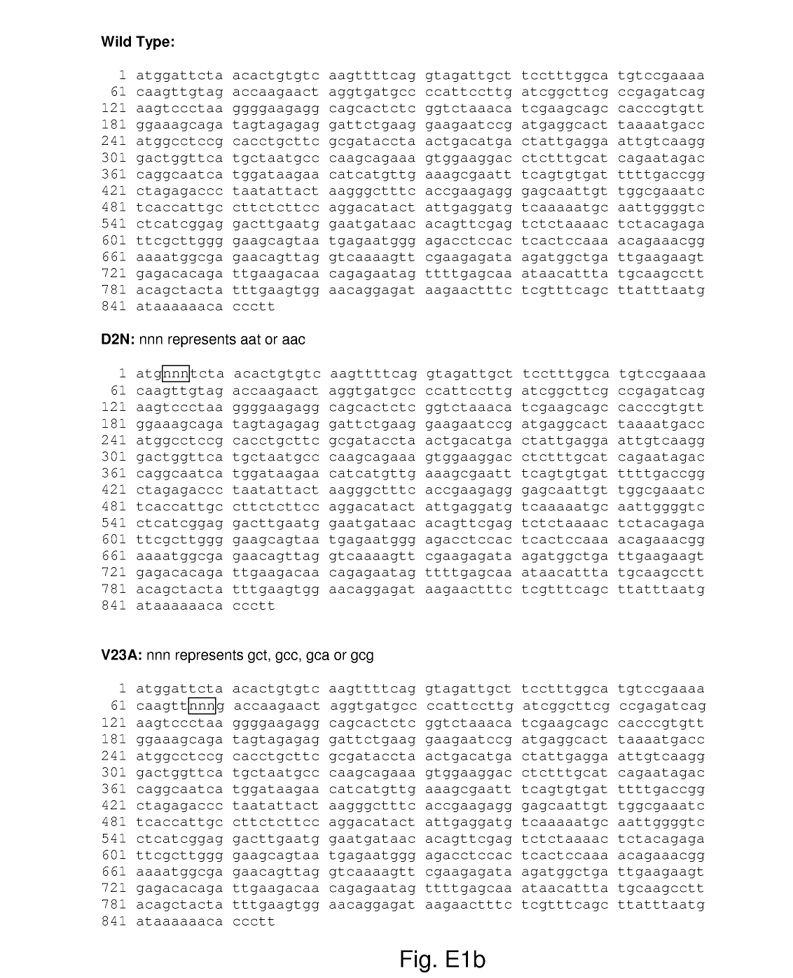

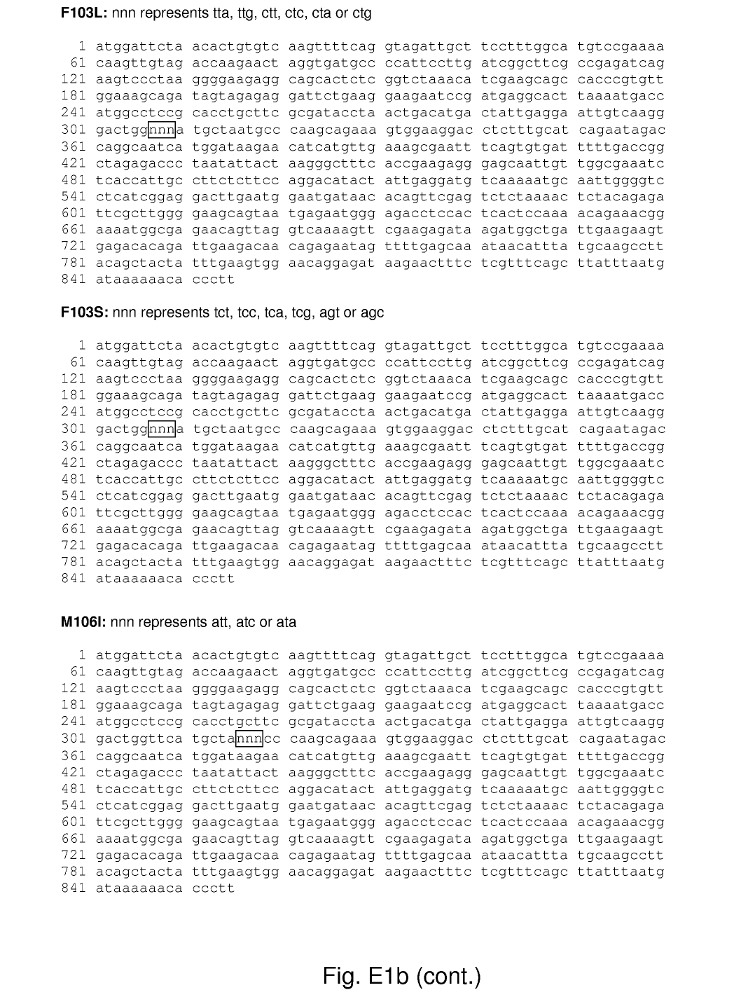

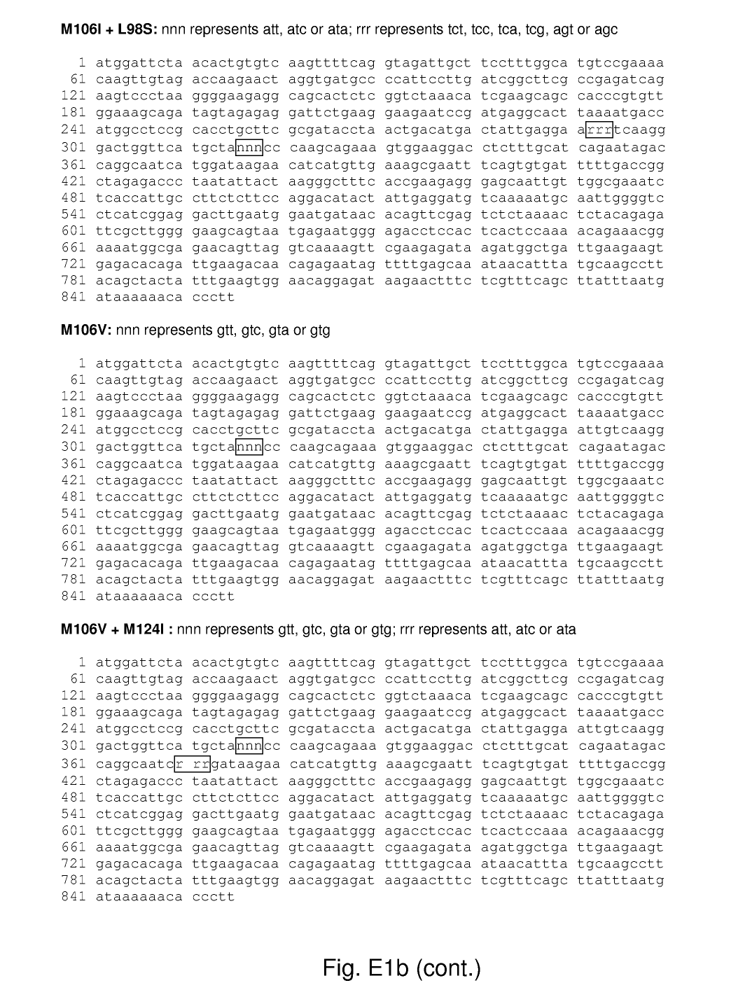

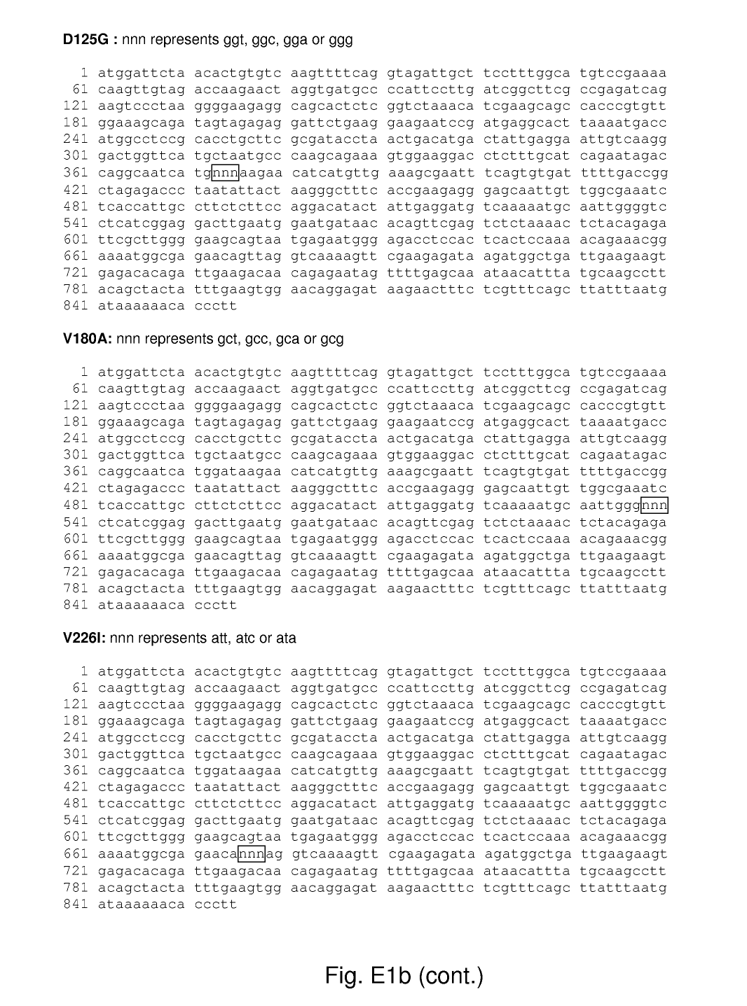

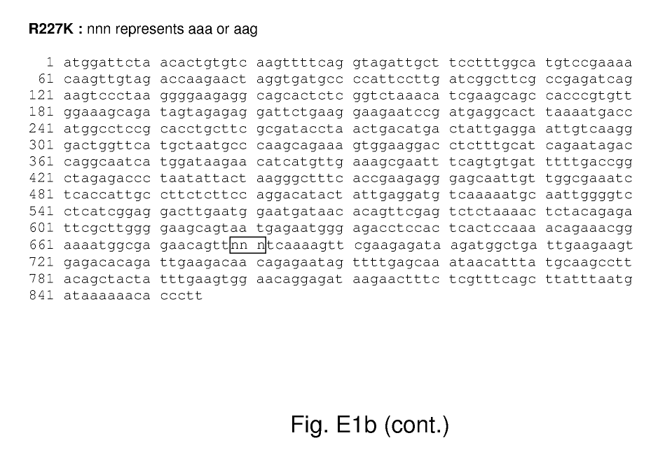

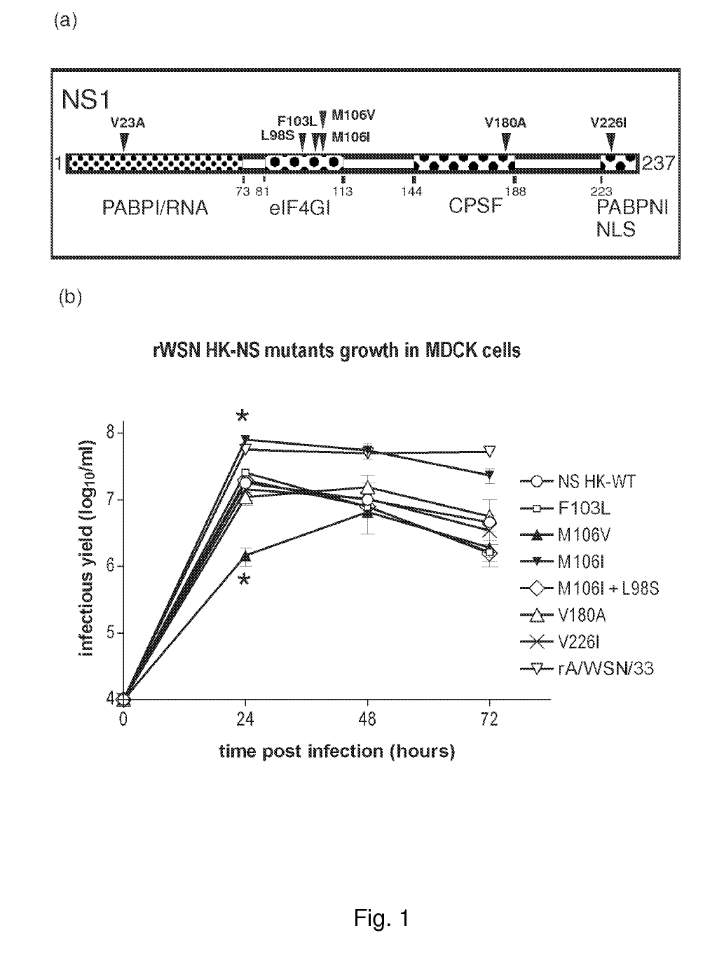

NS1 mutants were identified by sequencing the NS1 gene of 12 mouse-adapted clones derived from 7 independently passaged populations of A/HK/1/68. Six NS1 mutants were identified that possessed the following mutations: M106V, M106I, M106I+L98S, V180A, and V226I, in addition to the previously derived HKMA20 and HKMA20c variants (V23A and F103L mutations respectively (Brown, Liu, Kit, Baird, & Nesrallah, 2001)), (Table 2). All of the NS1 mutations—including V23A and F103L (Brown, Liu, Kit, Baird, & Nesrallah, 2001)—were under positive selection and thus adaptive as evidenced by their prevalence among clones from the same population except for the V226I mutation for which only one clone was sequenced. These mutations occurred within binding sites for RNA, translation initiation factors (eIF4GI and PABP1) and polyadenylation factors (CPSF and PABPN1) (FIG. 1a). The V180A NS1 mutation also induced a coincident S23P coding mutation in the overlapping NS2 gene (Table 2).

TABLE 2
NS1 gene mutations in MA variant clones of A/HK/1/68 (HK-wt) (H3N2).

| | NS1 | | | | | | | | | | NS2 | | |
|---|---|---|---|---|---|---|---|---|---|---|---|---|---|
| virus | 2 | 23 | 98 | 103 | 106 | 124 | 125 | 180 | 226 | 227 | 2 | 23 | 70 |
| HK-wt | D | V | L | F | M | M | D | V | V | R | D | S | G |
| HKMA20 (MA20) | — | A | — | — | — | — | — | — | — | — | — | — | — |
| HKMA20c (MA20C) | — | — | — | L | — | — | — | — | — | — | — | — | — |
| HK4MA21-1 (MA41) | — | — | — | — | V | — | — | — | — | — | — | — | — |
| HK4MA21-2 | — | — | — | — | V | — | — | — | — | — | — | — | — |
| HK4MA21-3 | — | — | — | — | V | I | — | — | — | — | — | — | — |
| HK5MA21-1 (MA51) | — | — | — | — | I | — | — | — | — | — | — | — | — |
| HK5MA21-2 | — | — | S | — | I | — | — | — | — | — | — | — | — |
| HK5MA21-3 (MA53) | — | — | S | — | I | — | — | — | — | — | — | — | — |
| HK6MA21-1 | — | — | — | — | — | — | — | — | — | — | — | — | — |
| HK6MA21-2 | — | — | — | — | — | — | — | A | — | — | — | P | — |
| HK6MA21-3 (MA63) | — | — | — | — | — | — | — | A | — | — | — | P | — |
| HK8MA21-1 | — | — | — | — | — | — | — | — | — | — | — | — | — |
| HK9MA21-3 | N | — | — | — | — | — | — | — | — | — | N | — | S |
| HK10MA21-3 (MA103) | — | — | — | — | — | — | — | — | I | — | — | — | — |
| HK11MA21-1 | — | — | — | — | — | — | G | — | — | — | — | — | — |
| HK11MA21-2 | — | — | — | — | — | — | — | — | — | K | — | — | — |
| PR/8/34 (mouse adapted) | — | — | — | S | I | — | — | — | — | — | — | — | — |

Using reverse genetics, recombinant viruses were constructed that expressed a HK wt or mutant NS1 gene with the remaining 7 gene segments derived from A/WSN/33, with the exception of V23A mutant that could not be rescued despite repeated attempts. All mutations were subsequently made on the HK/1/68(H3N2) genome and used for some analyses. In addition some mutations were made in the HKNS1 gene inserted into the PR/8/34(H1N1) virus. The growth of the rescued viruses in MDCK cells was determined by monitoring yield over 72 h following infection at moi of 0.02 (FIG. 1b). The HK-wt and all mutants had a similar peak yield at 24 h hr post infection except for the M106I and M106V mutants that had significantly greater and lesser yields respectively at this time. The M106I mutant was indistinguishable in yield from recombinant WSN (rWSN) virus.

### Example 2

### Adaptive NS1 Mutations Increased Protein Synthesis

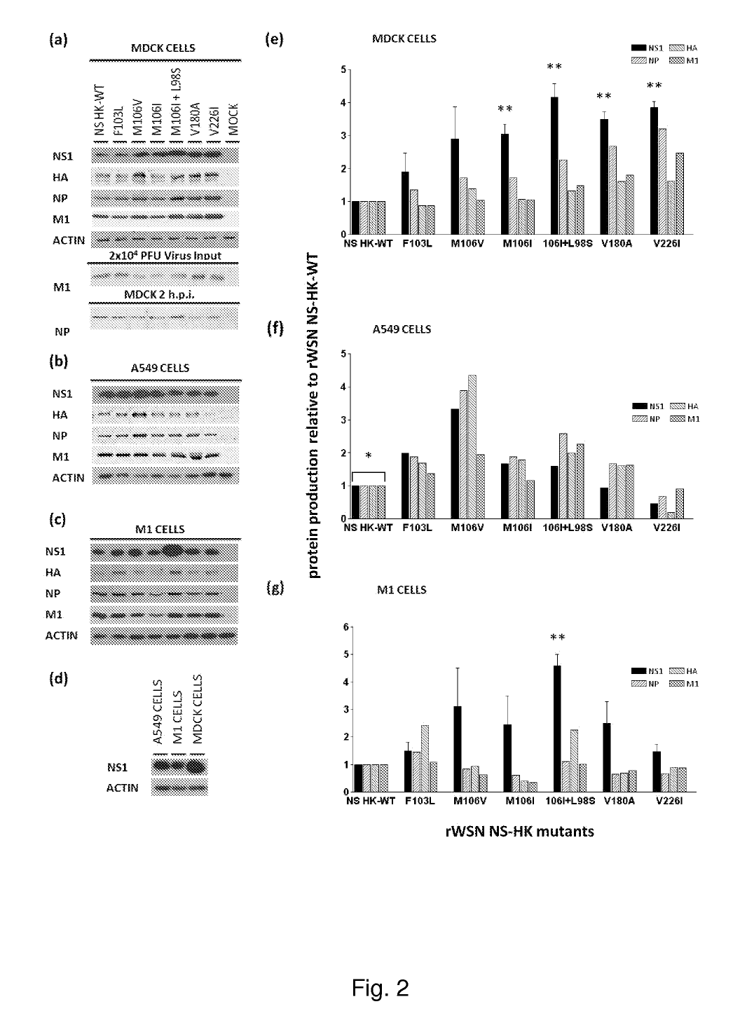

The occurrence and clustering of several mutations in the eIF4GI and PABP1 binding sites suggested that these mutations would affect protein synthesis. We therefore examined the relative ability of NS1 mutants to affect protein synthesis in MDCK cells at 8 h pi by Western blotting following infection at moi of 2. Protein blots were performed for NS1 proteins using rabbit anti-HK-wt NS1 serum as well as WSN HA, NP and M1 proteins using rabbit anti-A/WS/33 antiserum. Individual protein bands were quantified by densitometry and normalized relative to HK-wt and actin loading controls (FIGS. 2a and d). Using values from duplicate experiments all the rescued mutants except F103L and M106V produced significantly higher levels of NS1 protein (2 to 4 fold for M106I, M106I+L98S, V180A, and V226I) in MDCK cells (p<0.05 by t test). Western blotting analysis of the structural proteins showed elevated levels for the NP gene with lesser effects for the HA and M1 genes. Significant increases were seen for all mutants except F103L when comparing all four proteins among mutants (P<0.05 by t test). Control blots of 2×10⁴ pfu of stock virus for NP protein as well as at 2 hpi for M1 proteins showed that the levels of infection were comparable between strains and thus show that the differences in protein synthesis were not due to differences among input virus levels (FIG. 2a). The recombinant rWSN HK mutant viruses possessed identical structural proteins and thus establish infections with identical kinetics until the NS1 protein is expressed, with differences in the levels of proteins attributable to functions of the mutant genes. These data indicated that most of the mouse-adapted NS1 mutations increased protein synthesis but in a protein dependent manner with a greater effect seen for the NS1 and NP proteins.

We also measured proteins synthesis in human lung epithelium (A549) and mouse kidney epithelium (M1) cell lines because the original virus strain, A/HK/1/68, was a human clinical isolate that was then adapted to the mouse to generate NS1 variants. Protein synthesis was similarly enhanced in human A549 cells. Although the levels were not as high as seen in MDCK cells, all the mutants produced significantly more viral proteins (P<0.05 by t test) with the exception of the V226I mutant that induced lower levels of protein synthesis (FIGS. 2b and 2e).

NS1 mutant protein synthesis in mouse M1 cells was increased by 1.5 to 4 fold and thus was similar to MDCK but with less effect seen for the structural proteins at 8 hpi (FIGS. 2c and 20. In duplicate experiments NS1 protein expression was significantly greater only for the M106I+L98S mutant. The F103L and M106V mutants were seen to significantly increase NS1 gene expression in all 3 cell types when compared to HK-wt NS1 (p<0.05 by t test). Comparison blotting of rWSN HK-wt NS1 protein synthesis in the 3 host-cell types showed that translation in mouse cells was less than in human A549 and MDCK cells (FIG. 2d) suggesting an adaptive advantage for enhanced protein synthesis in mice. In general NS1 protein synthesis was elevated due to all NS1 mutations in all host cell types indicating that this was a host-independent phenotype.

### Example 3

### NS1 Mutations Increased IFN Induction

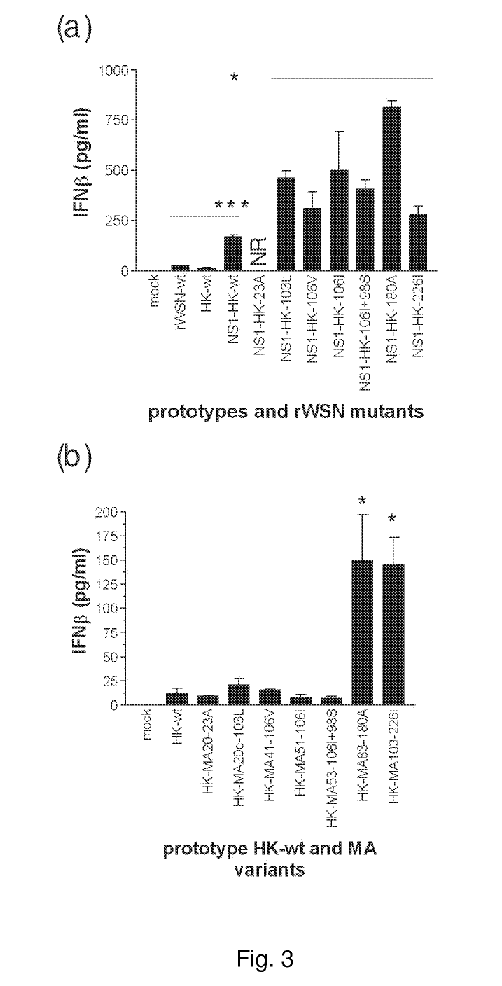

The reduced enhancement of structural protein synthesis in mouse cells relative to MDCK and A549 cells was unexpected given that these mutations were selected in mouse tissues. As NS1 proteins can differ in ability to inhibit IFN induction, the yield of IFNβ was measured by ELISA for M1 cell supernatants at 24 hpi infection at an moi of 2 with the WSN recombinants possessing each of the corresponding wt and mutant HK NS1 genes (except for rWSN-NS1-HK-V23A) as well as the WSN and HK-wt viruses as controls (FIG. 3a). The HK-wt and WSN viruses induced low levels of IFN (12 and 27 pg/ml respectively), whereas introduction of the HK-wt NS1 gene onto the WSN background resulted in a significant increase in IFNβ induction (168 pg/ml; 14 fold higher than HK-wt) indicating a role for non-NS1 genes in the control of IFN induction (P<0.05). Furthermore, each of the recombinant NS1 WSN mutants except M106I and M106V induced significantly more IFNβ than the HK-wt NS1 gene recombinant (P<0.05, FIG. 3a) with the 180A mutant inducing the greatest level of IFNβ. Because an increase in IFNβ induction was seen both with the introduction of the HK-wt NS1 on the foreign WSN backgrounds as well as increases due to each mutant gene, we measured the IFNβ induction of the MA variants that had originally acquired the mutant NS1 genes (FIG. 3b) and showed that most variants produced IFN yields that were not significantly different from HK-wt except for the V180A and V226I mutations that both induced significant higher levels of IFN(P<0.05) that were however less than the level induced when transferred to WSN. These data indicate that the role of the NS1 gene in IFNβ induction is influenced by both the presence of adaptive mutations as well as the nature of the genetic background.

### Example 4

### NS1 mutations increased Protein Synthesis Following IFN Pretreatment

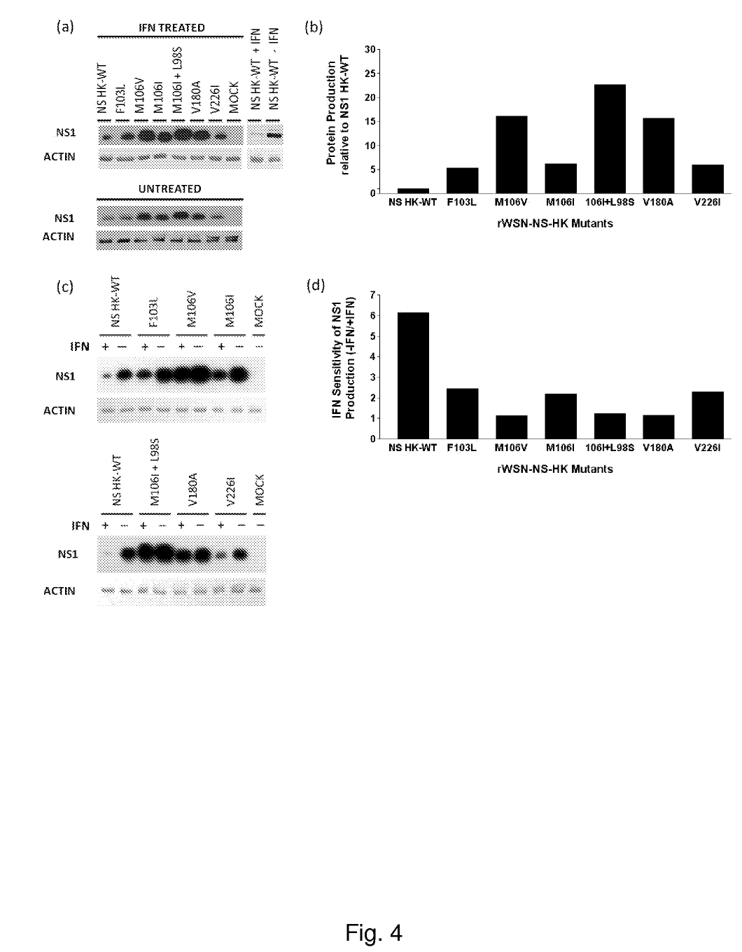

The observation that the WSN NS1-HK recombinants expressing adaptive NS1 mutations induced greater levels of IFN than rWSN HK-NS1-wt and yet expressed similar or higher levels of NS1 protein synthesis suggested that the NS1 mutations not only affected IFNβ induction but also enhanced the IFN resistance of protein synthesis. In order to directly assess resistance of protein synthesis to IFN, M1 cells were pretreated for 24 h with standardized amounts of 200 units of recombinant mouse IFNβ with subsequent measurement of protein synthesis as described in FIG. 1. We saw that all mutations significantly increased NS1 protein synthesis from 5 to 25 fold relative to HK-wt following IFN treatment indicating that all mutations possessed the property of IFN resistant NS1 protein synthesis (FIGS. 4a and b). Comparison of the ratio of protein synthesis for each NS1 gene following IFNβ pretreatment showed that HK-WT NS1 protein was reduced by a factor of 6 from its untreated levels the mutants were much more resistant being reduced by factors of 2.4 or 1.2 (FIGS. 4c and d). These data indicated that the mouse adaptive NS1 mutations all conferred IFN resistant protein synthesis.

### Example 5

### IFN Resistant Replication

We next examined the effect of IFN pretreatment on viral yield in mouse M1 cells. Mouse M1 were either untreated or IFN pretreated (200 U/ml mouse IFNβ) for 24 h before infection with rWSN-MA NS1 viruses at moi=2 followed by washing and overlay with medium containing 1 ug/ml trypsin with collection of virus at 24 hrs post infection. In control experiments input virus was not detectable in samples collected at t=0 h and incubated in parallel for 24 hrs indicating that virus detected in the samples was output virus.

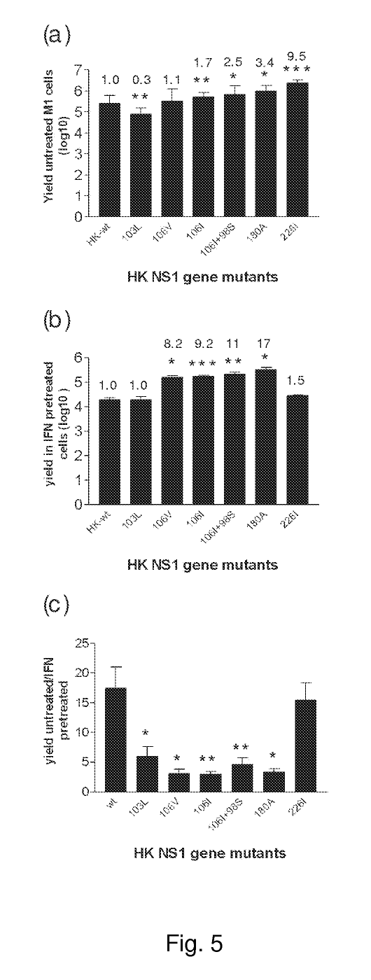

The yield from the single step growth cycle of the untreated samples showed that most mutations resulted in significantly increased replication with the exception of the M106V mutation that did not significantly change replication levels and the F103L mutation that significantly attenuated replication (FIG. 5a). Analysis of infectious yield from IFN pretreated M1 cells showed that 4 of the 6 mutants produced significantly higher yields of infectious virus, however no significant difference was seen for the F103L and V226I mutants. The 8 to 17 fold increased yield in the presence of IFN due to the M106V, M106I, M106I+L98S and V180A mutations indicates that they confer increased interferon resistance. The ratio of the yield without pretreatment relative to IFN pretreatment showed a significantly increased IFN resistance for viral replication for all mutants with a 17 fold difference in yield for HK-wt relative to a 3-6 fold difference for all mutants except V226I that demonstrated a 15 fold difference (FIG. 5c).

### Example 6

### CPSF Binding Ability of Mutant NS1 Proteins

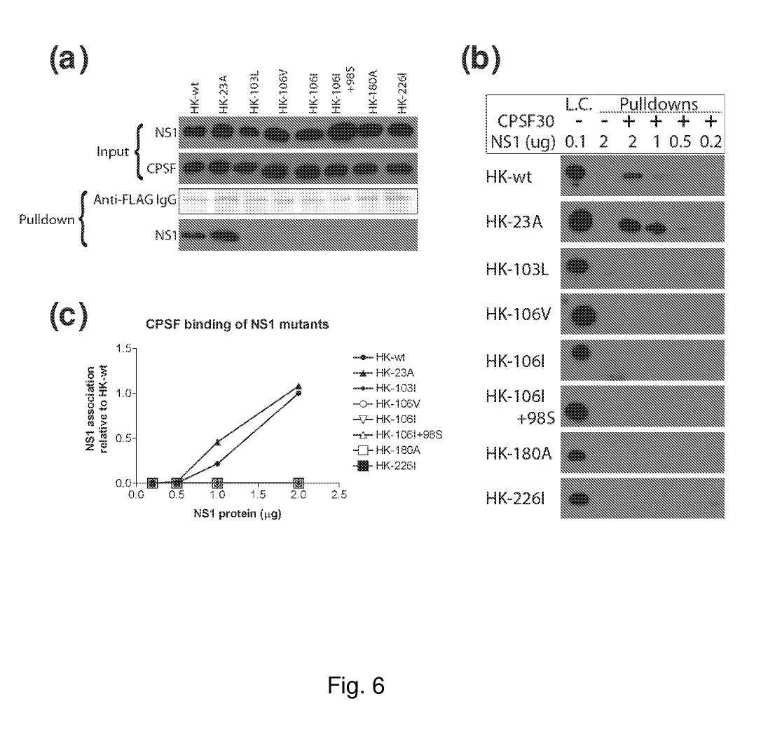

As NS1 proteins have been shown to bind CPSF and block post transcriptional processing and thus expression of IFN, we tested the ability of NS1 mutants to bind CPSF. Recombinant HK-wt and mutant NS1 proteins expressed in E. coli were mixed with FLAG tagged CPCSF protein expressed in 293T cells before immunoprecipitation with anti FLAG antibodies and quantification of NS1 binding by western blot. Using 1 μg aliquots of NS1 protein CPSF binding was detected for both wild-type and V23A mutants whereas NS1 binding was not detectable for the remaining mutants (FIG. 5a); the same data were obtained when using the F2F3 fragment of CPSF. Binding studies were repeated at NS1 concentration from 2 to 0.2 μg that show dose responsive binding for the HK-wt and V23A mutations that were not significantly different from each other but a total lack of binding under these conditions for all other mutants (FIG. 6 b and c). The loss of CPSF binding was consistent with the increased IFN induction due to these mutations.

### Example 7

### PABP1 Binding Ability of Mutant NS1 Proteins

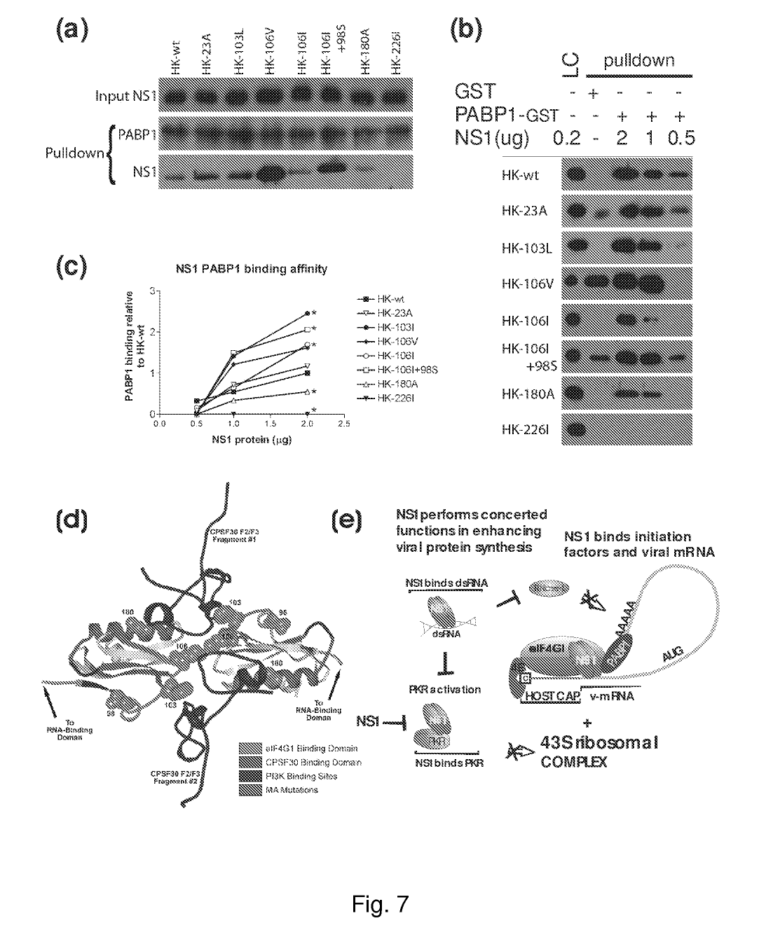

The ability of NS1 mutants to bind PABP1 was tested to see if this property was implicated in the mechanism of increased protein synthesis of the mutant NS1 proteins. Recombinant 6×His tagged HK-wt and mutant NS1 proteins were tested for binding to purified recombinant GST tagged PABP1 expressed in E. coli. GST tagged PABP1 was mixed with aliquots of NS1 proteins before pull-down with glutathione-tagged beads and quantification of NS1 binding by western blot. Using 1 μg aliquots of purified NS1 proteins and 3 μg of GST tagged PABP1 the NS1 mutants varied in their ability to bind PABP1 with both increased and decreased binding (FIG. 7a). Binding curves were generated using a range of NS1 concentrations, that showed increased PABP1 binding for the F103L, M106I, M106I+L98S, and M106V mutant proteins relative to HK-wt NS1 protein (p<0.05 by t test of duplicate experiments) whereas others were either unchanged in binding as seen for the 23A mutant or were reduced in binding for 180A and 226 I (p<0.05 by t test) (FIGS. 7b and c).

### Example 8

### Analysis of the Effect of NS1 Mutations on Viral Growth In Vitro

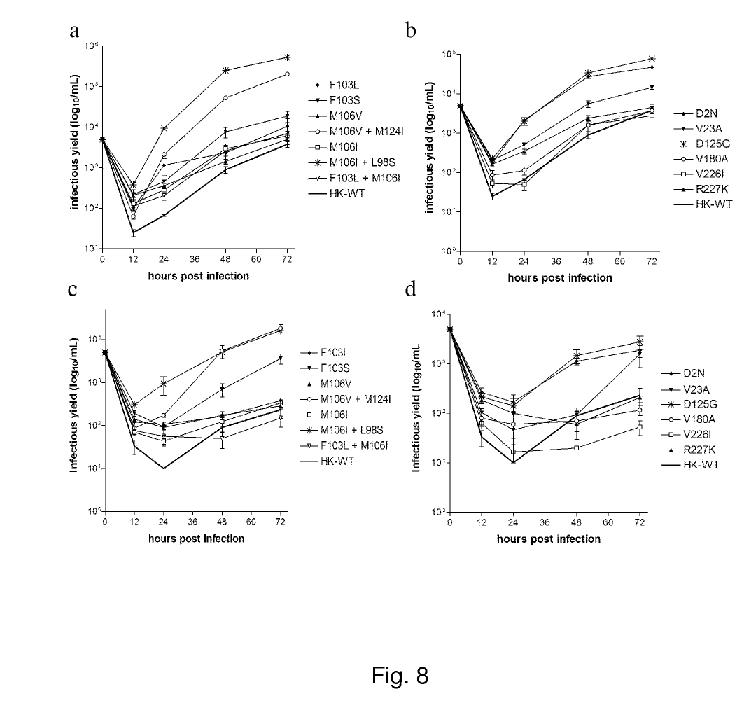

The effect of the NS1 mutations on viral growth was studied by infecting mouse kidney epithelial (M1) cells with recombinant A/Hong Kong/1/1968 viruses expressing wild-type or mutant NS1 genes (D2N, V23A, F103L, F103L+M106I, F103S, M106I, M106I+L98S, M106V, M106V+M124I, D125G, V180A, V226I, and R227K). Parallel experiments were carried out using M1 cells that were left untreated and M1 cells pretreated with 1000 U/mL murine IFNβ for 24 hours. The results are shown in FIG. 8 for both untreated M1 cells (a, b) and pretreated cells (c, d). Cells were infected at a MOI of 0.02 with recombinant A/Hong Kong/1/1968 viruses expressing wild-type Hong Kong NS1 or mutant NS1 genes as indicated. Following infection, the cells were supplemented with Serum-Free MEM in the presence of 0.5 μg/mL trypsin. Supernatant samples were taken at 12, 24, 48 and 72 hours post infection, and assayed for viral yield by plaque assay in MDCK cells in triplicate. Values are shown as means plus and minus standard deviation. The results show that the mouse adapted NS1 mutations enhance viral growth in vitro irrespective of IFN pretreatment.

### Example 9

### NS1 Mutations Enhance Viral Virulence

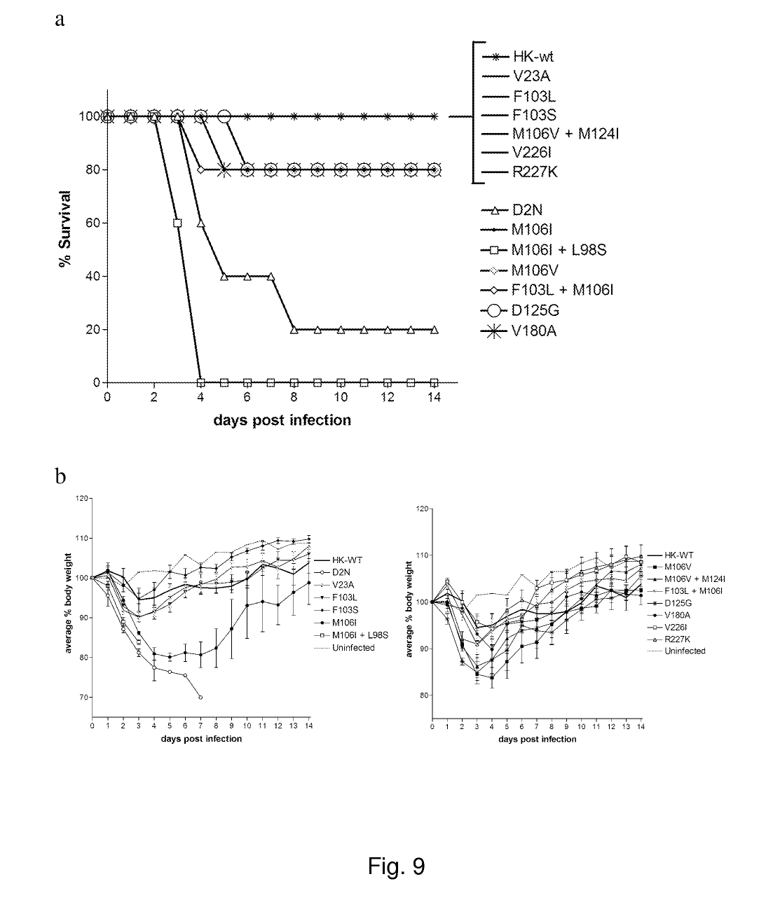

The effect of NS1 mutations on viral virulence was studied by monitoring progression of disease in CD-1 mice infected with viruses expressing either wild-type or mutant NS1 genes (D2N, V23A, F103L, F103L+M106I, F103S, M106I, M106I+L98S, M106V, M106V+M124I, D125G, V180A, V226I, and R227K). Mice were intranasally infected with a 5×10⁶ dose of recombinant A/Hong Kong/1/1968 viruses expressing wild-type Hong Kong NS1 or mutant NS1 genes (5 mice per virus). Mice were then monitored for progression of disease for 14 days following infection. Results are shown in FIG. 9, including (a) percent survival, and (b) average percent body weight, and indicate that mouse adapted NS1 mutations enhance virulence in the mouse. Values are shown as the means plus and minus the standard deviation.

### Example 10

### In Vivo Analysis of the Effect of NS1 Mutations on Immune Response in Mice

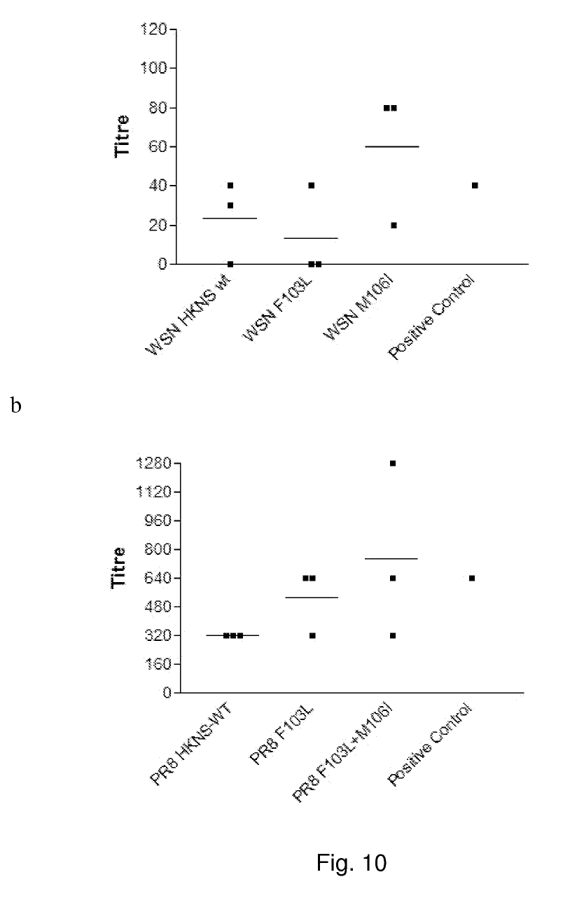

The effect of NS1 mutations on immune response was tested using two influenza strains (WSN and PR8) possessing either wild type or mutant NSI genes (F103L; and M106I or F103L+M106I mutants). Mice were infected with the same dosages of live viruses (groups of mice each were infected with 1×10⁵ pfu given intranasally), with blood collection 21 days after infection. Sera were treated with neuraminidase and 56° C. to inactivate inhibitors before HAI assay using 8 HA units of WSN or PR8 viruses. Immune responses for WSN H1N1 that possessed the HK NS1 genes were tested for viruses possessing HK-wild type (WSN HKNS wt), HK F103L mutant, and HKM106I mutant. These viruses induce a very poor immune response, although average antibody titers measured by HAI were higher for the HKM106I mutant (values <20 are recorded as 0). See FIG. 10 (a). Immune responses were also tested for the mutations in the PR8 virus backbone for viruses possessing HK-wild type (PR8HKNS wt), HK F103L mutant (PR8HKNS F103L), and HK F103L+M106I mutant (PR8HKNS F103L+M106I). Both the HK F103L mutant and HK F103L+M106I mutant resulted in increased average antibody levels (See FIG. 10 (b)). These results indicate that the adapted NS1 mutations increase average antibody levels relative to wild type in mice, although this effect could not be seen for the F103L mutation in the WSN viral strain tested. This may be due to the fact that the WSN strain has an ineffective growth property with respect to immune response, and thus the effect of this mutation might not have been detectable in this assay with this low growth strain. This effect was, however, observed for the F103L mutation in the PR8 viral strain, which possesses effective growth properties, as well as for the M106I and F103L+M106I mutants in the respective viral strains used in the study.

### Example 11

### Mutations in the Influenza a Virus NS1 Gene Increase RNA Polymerase Activity

The role of NS1 mutations in RNA polymerase activity was measured because NS1 is known to bind to influenza viral RNA-polymerase and a panel of 6 mutant have been shown to increase influenza virus gene expression.

Plasmids: Plasmids expressing each of the HK-WT PB1, PB2, PA, and NP proteins and each of the NS1 mutations, listed Table 1, were constructed under control of the CMV and POL1 promoters for use in luciferase minigenome expression assay.

### Luciferase Assay Procedure:

1. 293T cells were grown in a volume of 150 μl of DMEM supplemented with 10% FCS in 96-well plates.
2. When the cells were confluent to 80%, proceed to carry out transfection.
3. Dilute the plasmids into 0.03 μg/μl (note each plasmid adds 0.06 μg) adding PB2, PB1, PA NP, and phPolI-luc-NP, PRL-SV40, and NS1 protein plasmid
4. To each well add 0.5 μl Lipofectamine 2000 (0.5 μl+49.5 μl Opti-MEM). Total volume: plasmids+Lipofectamine 2000+Opti-MEM=100 μl.
5. After 16 h, replace the medium with Opti-MEM
6. At 24 h after transfection, assay for luciferase activity by using the Dual-Glo™ Luciferase assay system detection kit (Promega).

TABLE 3
List of A/HK/1/68- mouse adapted variant NS1 adaptive
mutations derived from mouse adaption.

HKNS
HKNS V23A
HKNS F103L
HKNS D2N
HKNS F103L M106I
HKNS M106V
HKNS M106V M124I
HKNS M124I
HKNS D125G
HKNS R227K
HKNS M106I
HKNS M106I L98S
HKNS V180A
HKNS V226I

TABLE 4
List of Polymerase assays.

HK(3P + NP)
HK(3P + NP) + PLLB-HKNS
HK(3P + NP) + PLLB-HKNS V23A (MA20)
HK(3P + NP) + PLLB-HKNS F103L (MA20C)
HK(3P + NP) + PLLB-HKNS D2N (MA93)
HK(3P + NP) + PLLB-HKNS F103L M106I
HK(3P + NP) + PLLB-HKNS M106V (MA41)
HK(3P + NP) + PLLB-HKNS M106V M124I
HK(3P + NP) + PLLB-HKNS M124I
HK(3P + NP) + PLLB-HKNS D125G (MA102)
HK(3P + NP) + PLLB-HKNS R227K (MA112)
HK(3P + NP) + PLLB-HKNS M106I (MA51)
HK(3P + NP) + PLLB-HKNS M106I L98S (MA53)
HK(3P + NP) + PLLB-HKNS V180A (MA63)
HK(3P + NP) + PLLB-HKNS V226I (MA103)

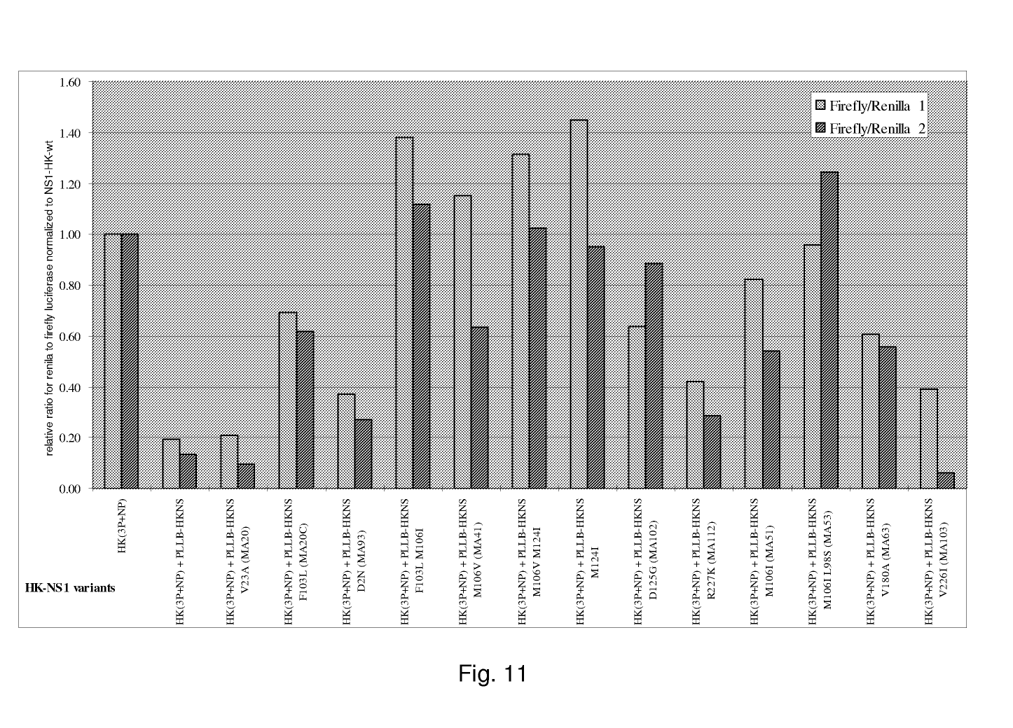

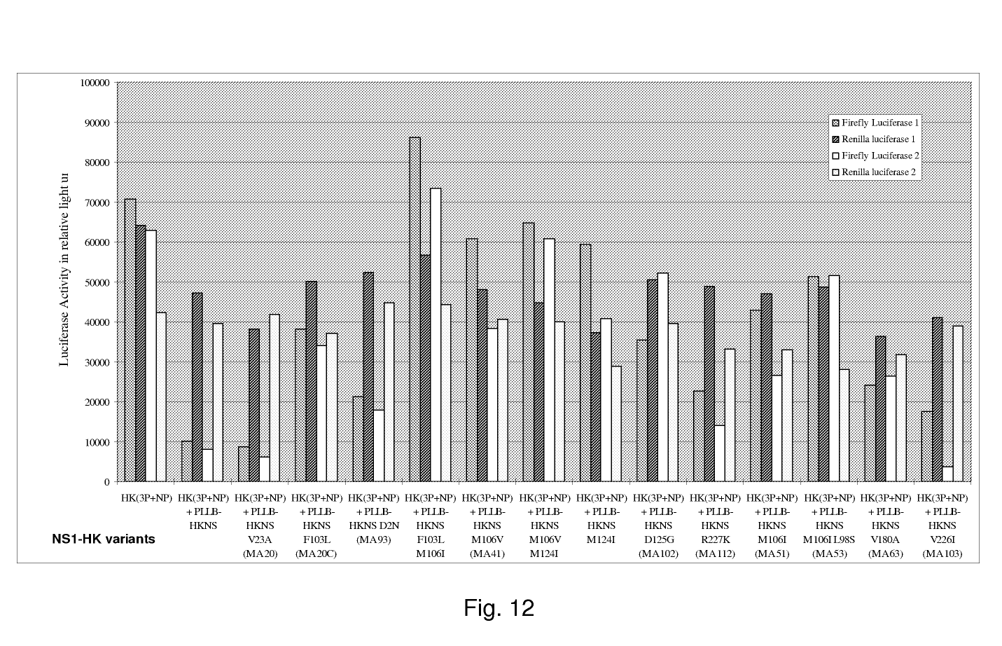

FIG. 11 shows the effect of the NS1 mutations on RNA polymerase activity normalized to A/HK/1/68 wild-type activity. Polymerase activity was measured by comparison of renilia luciferase activity from a plasmid driven with a CMV promoter relative to a firefly luciferase construct that possessed the 5′ and 3′ ends of NP genome segment. The control assay contains polymerase components only: PB1, PB2 PA and NP and the reference wild type NS1 sample has NS1-HK-wt added for reference to the HK-mouse adapted mutant NS1 plasmids. All samples were normalized to the control (HK(3P+NP)). The wild type segment 8 has an inhibitory effect on RNA polymerase activity that it reversed by the F103L, D2N, M106I, M106V, M124I, D125G, R227K, L98S, V180A, but was not increased by the V23A or V226I. The raw data are shown in FIG. 12.

The wild type NS1 protein has a 5 fold inhibitory effect on RNA polymerase activity. Polymerase activity was enhanced due to the following NS1 mutations: F103L, D2N, M106I, M106V, M124I, D125G, R227K, L98S, V180A. In contrast the V23A or V226I mutations affect on transcription did not differ from that of NS1-HK-wild type.

This data indicates that derived NS1 mutants can enhance gene expression by increasing transcription that will contribute to the increased protein synthesis seen for these mutations. Specifically the described NS1 mutations serve as regulators of RNA polymerase activity.

### Discussion of Mechanisms

The principle function of a virus is to replicate; adaptation to a new host involves the selection of mutations that increase replication through functional modification of interactions with viral or host components. The experimental mouse model was used to derive a group of 7 adaptive mutations in the NS1 gene that increased viral replication and gene expression and furthermore increase these properties in the presence to IFN. In general it is expected that adaptive mutations will increase binding to factors where binding favors viral replication or conversely decrease binding to factors where decreased binding favors replication, such as to inhibitors, as shown for a mammalian inhibitor of avian PB2 possessing 627E that escapes inhibition on adaptation to 627K {Mehle, 2008 454/id}. At the biochemical level, all mutations in the effector domain caused a loss of CPSF binding and 4 of 6 (F103L, M106V, M106I and L98S) increased binding to PABP1. The mutations increased protein synthesis in a host independent manner. This is consistent with the observation that the NS1 binding sites in both CPSF and PABP1 of human and mouse are identical indicating that adaptive changes were not in response to differences in the host binding sites but rather have altered the extent of NS1 protein interactions to enhance functions.

Biological and biochemical studies thus demonstrated that all mutations were adaptive and furthermore were multifunctional causing both gain and loss of function associated with IFN induction and IFN resistant protein synthesis and replication.

### Mechanism of Increased IFN Resistant Protein Synthesis

The NS1 protein is known to increase translation of viral mRNA or mRNA engineered to possess the 5′ noncoding region of influenza virus mRNA. Protein expression is enhanced in the range of 5 to 100 fold for M1 protein (de la, Fortes, Beloso, & Ortin, 1995), by NS1 binding to viral mRNA, PABP1 (Burgui, Aragon, Ortin, & Nieto, 2003) and eIF4G1 (Aragon et al., 2000). The NS1 binding site in eIF4G1 is adjacent to the PABP1 binding site and thus is compatible with NS1 binding to both eIF4G1 and PABP1 simultaneously as shown by Bergui et al. (2003). In addition the NS1 protein binding sites for eIF4GI and PABP1 are partially overlapping where the eIF4GI binds to amino acid region 81-113 and the PABP1 has a minimal essential binding site from 1-73 but with increased binding conferred by the 74-150 aa region {Burgui, 2003 217/id}. Increased PABP1 binding is consistent with a mechanism of increased protein synthesis due to increased NS1 mediated recruitment of translation initiation complexes to viral mRNA. Whereas we did not test eIF4GI binding, the increased binding of PABP1 to NS1 may increase the overall activity of the initiation complex through allosteric effects as seen for mutations that increase binding between individual components of the initiation complex, see review of (Prevot et al., 2003). This mechanism is also consistent with the greatest observed effects on translation enhancement seen for the NS1 protein itself due to autocatalytic effects of NS1 protein on NS1 mRNA due to proximity effects. As NS1 protein is known to bind dsRNA and reduce activation of IFN effectors, such as RNAse L (Min & Krug, 2006) and PKR (Tan & Katze, 1998) or by binding PKR directly (Li et al., 2006; Min, Li, Sen, & Krug, 2007), it is possible that increased binding to dsRNA or PKR due to adaptive mutations would also enhance protein synthesis as proposed in FIG. 7e.

### IFN Resistance and Protein Synthesis

The data support a mechanism of IFN resistance that involves enhanced IFN resistant protein synthesis. Whereas all the rescued mutants induced greater accumulation of viral proteins both in the presence and absence of IFN, not all mutants demonstrated IFN resistant viral replication. The V226I mutant was an exception suggesting that enhanced gene expression was not sufficient in itself to mediate IFN resistance. Whereas the V226I mutant was the most adaptive in M1 cells, increasing yield by 8.5 fold, this mutation may enhance gene expression through mechanisms that that are distinct from the other mutants.

NS1 Protein Increases IFNβ Induction

The greatest increases in protein synthesis were seen in infected canine kidney MDCK cells with lesser effects seen for human lung epithelium (A549) and mouse kidney M1 cells. The lower level of protein synthesis seen in the mouse cells was associated with increased IFN induction, indicating a decreased ability to inhibit IFN induction by NS1 mutants rescued onto the WSN backbone. This was a cryptic property that emerged when either the HK-wt NS1 or HK mutants were expressed on the foreign WSN background, as the parental HK-wt and WSN as well as each of the mouse adapted NS1 variants were seen to be low inducers of IFNβ in M1 cells (FIG. 3b). This indicated that all the NS1 mutations were selected in a genetic background that maintained an ability to inhibit IFN induction. The loss of ability to inhibit IFN induction when the HK-NS1 was transferred into WSN indicates that interactions were lost or disrupted due to genetic differences between the HK and WSN backbones. Increased IFNβ was associated with a loss of binding to CPSF where CPSF binding results in inhibition of IFNβ and other host gene expression (Nemeroff et al., 1998). More recently NS1 protein has been shown to bind CPSF in a complex with polymerase genes and furthermore that this results in epistatic effects that suppress the loss of CPSF binding due to the 103L+106I mutations in NS1-HK/156/97H5N1 (Kuo & Krug, 2009). The fact that mutant NS1 proteins that cannot bind CPSF in isolation can, however suppress IFN induction on their native genetic backgrounds suggests that CPSF binding may require a complex of HK viral polymerase and NS1 proteins. Thus there are distinct functional roles for free versus complexed NS1 proteins.

Given that we have demonstrated an increased synthesis of NS1 protein due to these mutations there will be higher levels of both complexed and free forms of NS1 to interact with ligands in either of the states. Furthermore whereas there will be high and effective levels of complexed proteins that interact with CPSF there will be higher levels of free NS1 that can now bind to other ligands in the absence of competition for CPSF binding.

Alternatively to restoration of CPSF binding by NS1 protein interactions, we cannot exclude the possibility that the balance of NS1 functions that provide inhibition of IFN induction may be different for HK NS1 protein and may not require CPSF binding because other viruses such as PR/8/34 (H1N1) have been shown to have lost CPSF binding due to mutations at positions 103 and 106 (S+I) and yet block IFN induction due to an ability to prevent pretranscriptional activation of IRF-3, NFκB and c-Jun/ATF-2 (reviewed (Hale, Randall, Ortin, & Jackson, 2008)). PR8/NS1 may mediate this in part by associating with the cytoplasmic RIG-I sensor complex a property that is not shared with A/Texas/36/91-NS1 (H1N1) which binds poorly to this complex ((Kochs et al., 2007)). Future studies are needed to assess the role of the adaptive HK-NS1 mutations on interactions with RIG-I and other factors that control the IFN response.

### Increased Protein Synthesis and Gene Dosage

The fact that these NS1 mutations function to increase protein synthesis means that there will be greater levels of NS1 and other viral proteins to result in higher than normal levels of function(s) associated with these genes due to gene dosage effects. While increased protein synthesis will enhance replication up to a point, at a given level some functions will be supra-optimal and inhibit replication. It may be that mutations that enhance protein synthesis are only adaptive if they can down regulate functions that cannot themselves be increased without deleterious effects. For example, CPSF binding may be a function that cannot be increased to higher levels because this would inhibit host gene expression to levels that are inhibitory to viral replication such as seen for chemical inhibitors of host POL II transcription such as α-amanatin or actinomycin D {Mark, 1979 442/id}. Influenza A virus has a functional requirement for host cell transcripts that are used as sources of cap structures to prime viral mRNA {Engelhardt, 2006 244/id}. Influenza has also been shown to employ PI3K activation to increase replication {Shin, 2007 236/id} and excessive inhibition of host transcription may be deleterious with respect to achieving effective PI3K activation.

As NS1 partitions to both the nucleus and cytoplasm with distinct roles in each of these locations, the described NS1 mutations may effect the timing or extent of cellular location that could further influence the function of mutant NS1 proteins. Changed localization would be further modulated by altered functions that affect the balance of functions of NS1 mutants

### Adaptive Domains in NS1

Mutations could be divided into 3 groups on the basis of their location and properties. Most of the mutations (4 of 7) resided in the proximal end of the “effector domain” in the middle of the eIF4GI binding site between aa positions 98 to 106, and were all shown to confer increased protein synthesis, IFN resistance, increased PABP1 binding and a loss of CPSF binding. These mutations were distributed along a band that is outside, but bisects, the 2 CPSF binding pockets (FIG. 7d) and where 103F and 106I have been shown to be essential in stabilizing CPSF binding through hydrophobic interactions; which was confirmed in our studies {Das, 2008 440/id}. Position 106M of both NS1 chains also interact to stabilize the dimmer. Convergent evolution was observed at position 106 (to I or V) demonstrating strong selection for adaptation at this site. Furthermore sequential mutations were seen at position 106I followed by 98S that both significantly increased proteins synthesis (FIGS. 1a and d; P<0.05).

Two mutations were observed at the distal region of the effector domain within the CPSF and PABPN1 binding sites. Both these mutations increased proteins synthesis in the presence or absence of IFN however only the 180 mutant enhanced replication in the presence of IFN. Both mutants abrogated CPSF binding and it is possible that the V226I mutation reduces PABPN1 binding that would also function to reduce inhibition of host gene expression, although this awaits further analysis.

The V23A mutation was the most distinct as the only mutation in the RNA and PABP1 binding domain, (located distally, in a loop that joins the first 2 α-helices {Bornholdt, 2008 443/id}), that did not affect binding to either CPSF or PABP1, nor could it be rescued onto the WSN backbone indicating requirements for additional protein interactions or functions.

### Adaptive NS1 Mutations Increased IFN Induction and IFN Resistance

Under standardized conditions of infection involving IFN pretreatment, NS1 mutations were all shown to increase protein synthesis relative to HK-wt indicating that these mutations enhanced IFN antagonism. This strongly suggested that the selective force during adaptation was for IFN resistance and that this was mediated by mutations that increased protein synthesis in the presence of IFN. The 92E mutation has been shown to be necessary for the IFN resistance property of A/HK/156/97-NS1 {Seo, 2002 78/id} and maps near the cluster of mutations between 98 and 106 further supporting the functioning of this region as an IFNβ resistance domain.

Although the loss of CPSF binding and the decrease in PABP1 binding were mapped to the 180A mutation using recombinant proteins, we could not unambiguously map the mutation at 180A to the properties because this substitution (T539C) also changed the coding of NS2 gene (S23P) and we cannot exclude the possibility that the NS2 mutations contributed to these phenotypes. Future experiments are needed to clarify this situation however it is not possible to mutate these sites independently of each other.

### CONCLUSION

NS1 mutations selected on serial passage were adaptive for replication and demonstrated increased IFNβ resistant protein synthesis and replication in IFNβ pretreated cells. Enhanced proteins synthesis was mechanistically associated with increased binding to PABP1 and increased IFNβ induction was associated with a loss of binding to CPSF. Adaptive NS1 mutations mediated replicative gains-of-function mutations that increased viral gene expression and IFN resistance, but were also associated with loss of function, seen as decreased ability to block host gene expression that also implicated NS1 gene interaction with other non-NS1 genes.

Although this invention is described in detail with reference to preferred embodiments thereof, these embodiments are offered to illustrate but not to limit the invention. It is possible to make other embodiments that employ the principles of the invention and that fall within its scope as defined by the claims appended hereto. All scientific and patent publications cited herein are hereby incorporated in their entirety by reference.

### REFERENCES

1. Aragon, T., de la, L. S., Novoa, I., Carrasco, L., Ortin, J. & Nieto, A. (2000). Eukaryotic translation initiation factor 4GI is a cellular target for NS1 protein, a translational activator of influenza virus. Mol Cell Biol 20, 6259-6268.
2. Brown, E. G. (1990). Increased virulence of a mouse-adapted variant of influenza A/FM/1/47 virus is controlled by mutations in genome segments 4, 5, 7, and 8. J Virol 64, 4523-4533.
3. Brown, E. G. & Bailly, J. E. (1999). Genetic analysis of mouse-adapted influenza A virus identifies roles for the NA, PB1, and PB2 genes in virulence. Virus Res 61, 63-76.
4. Brown, E. G., Liu, H., Kit, L. C., Baird, S. & Nesrallah, M. (2001). Pattern of mutation in the genome of influenza A virus on adaptation to increased virulence in the mouse lung: identification of functional themes. Proc Natl Acad Sci USA 98, 6883-6888.
5. Burgui, I., Aragon, T., Ortin, J. & Nieto, A. (2003). PABP1 and eIF4GI associate with influenza virus NS1 protein in viral mRNA translation initiation complexes. J Gen Virol 84, 3263-3274.
6. de la, L. S., Fortes, P., Beloso, A. & Ortin, J. (1995). Influenza virus NS1 protein enhances the rate of translation initiation of viral mRNAs. J Virol 69, 2427-2433.
7. Gabriel, G., Dauber, B., Wolff, T., Planz, O., Klenk, H. D. & Stech, J. (2005). The viral polymerase mediates adaptation of an avian influenza virus to a mammalian host. Proc Natl Acad Sci USA %20; 102, 18590-18595.
8. Garcia-Sastre, A. (2006). Antiviral response in pandemic influenza viruses. Emerg Infect D is 12, 44-47.
9. Garcia-Sastre, A. & Biron, C. A. (2006). Type 1 interferons and the virus-host relationship: a lesson in detente. Science 312, 879-882.
10. Garcia-Sastre, A., Egorov, A., Matassov, D., Brandt, S., Levy, D. E., Durbin, J. E., Palese, P. & Muster, T. (1998). Influenza A virus lacking the NS1 gene replicates in interferon-deficient systems. Virology %20; 252, 324-330.
11. Hale, B. G. & Randall, R. E. (2007). PI3K signalling during influenza A virus infections. Biochem Soc Trans 35, 186-187.
12. Hale, B. G., Randall, R. E., Ortin, J. & Jackson, D. (2008). The multifunctional NS1 protein of influenza A viruses. J Gen Virol 89, 2359-2376.
13. Hu, Y. W., Rocheleau, L., Larke, B. & other authors (2005). Immunoglobulin mimicry by Hepatitis C Virus envelope protein E2. Virology %20; 332, 538-549.
14. Keleta, L., Ibricevic, A., Bovin, N. V., Brody, S. L. & Brown, E. G. (2008). Experimental evolution of human influenza virus H3 hemagglutinin in the mouse lung identifies adaptive regions in HA1 and HA2. J Virol 82, 11599-11608.
15. Kochs, G., Garcia-Sastre, A. & Martinez-Sobrido, L. (2007). Multiple anti-interferon actions of the Influenza A virus NS1 protein. J Virol.
16. Krug, R. M., Yuan, W., Noah, D. L. & Latham, A. G. (2003). Intracellular warfare between human influenza viruses and human cells: the roles of the viral NS1 protein. Virology 309, 181-189.
17. Kuo, R. L. & Krug, R. M. (2009). Influenza a virus polymerase is an integral component of the CPSF30-NS1A protein complex in infected cells. J Virol 83, 1611-1616.
18. Li, S., Min, J. Y., Krug, R. M. & Sen, G. C. (2006). Binding of the influenza A virus NS1 protein to PKR mediates the inhibition of its activation by either PACT or double-stranded RNA. Virology 349, 13-21.
19. Marion, R. M., Aragon, T., Beloso, A., Nieto, A. & Ortin, J. (1997a). The N-terminal half of the influenza virus NS1 protein is sufficient for nuclear retention of mRNA and enhancement of viral mRNA translation. Nucleic Acids Res 25, 4271-4277.
20. Marion, R. M., Zurcher, T., de la, L. S. & Ortin, J. (1997b). Influenza virus NS1 protein interacts with viral transcription-replication complexes in vivo. J Gen Virol 78, 2447-2451.
21. Mibayashi, M., Martinez-Sobrido, L., Loo, Y. M., Cardenas, W. B., Gale, M., Jr. & Garcia-Sastre, A. (2007). Inhibition of retinoic acid-inducible gene I-mediated induction of beta interferon by the NS1 protein of influenza A virus. J Virol 81, 514-524.
22. Min, J. Y. & Krug, R. M. (2006). The primary function of RNA binding by the influenza A virus NS1 protein in infected cells: Inhibiting the 2′-5′ oligo (A) synthetase/RNase L pathway. Proc Natl Acad Sci USA 103, 7100-7105.
23. Min, J. Y., Li, S., Sen, G. C. & Krug, R. M. (2007). A site on the influenza A virus NS1 protein mediates both inhibition of PKR activation and temporal regulation of viral RNA synthesis. Virology.
24. Nemeroff, M. E., Barabino, S. M., Li, Y., Keller, W. & Krug, R. M. (1998). Influenza virus NS1 protein interacts with the cellular 30 kDa subunit of CPSF and inhibits 3′ end formation of cellular pre-mRNAs. Mol Cell 1, 991-1000.
25. Neumann, G., Watanabe, T., Ito, H. & other authors (1999). Generation of influenza A viruses entirely from cloned cDNAs. Proc Natl Acad Sci USA 96, 9345-9350.
26. Noah, D. L., Twu, K. Y. & Krug, R. M. (2003). Cellular antiviral responses against influenza A virus are countered at the posttranscriptional level by the viral NS1A protein via its binding to a cellular protein required for the 3′ end processing of cellular pre-mRNAS. Virology 307, 386-395.
27. Prevot, D., Darlix, J. L. & Ohlmann, T. (2003). Conducting the initiation of protein synthesis: the role of eIF4G. Biol Cell 95, 141-156.
28. Shin, Y. K., Liu, Q., Tikoo, S. K., Babiuk, L. A. & Zhou, Y. (2007). Influenza A virus NS1 protein activates the phosphatidylinositol 3-kinase (PI3K)/Akt pathway by direct interaction with the p85 subunit of PI3K. J Gen Virol 88, 13-18.
29. Talon, J., Horvath, C. M., Polley, R., Basler, C. F., Muster, T., Palese, P. & Garcia-Sastre, A. (2000). Activation of interferon regulatory factor 3 is inhibited by the influenza A virus NS1 protein. J Virol 74, 7989-7996.
30. Tan, S. L. & Katze, M. G. (1998). Biochemical and genetic evidence for complex formation between the influenza A virus NS1 protein and the interferon-induced PKR protein kinase. J Interferon Cytokine Res 18, 757-766.
31. Twu, K. Y., Noah, D. L., Rao, P., Kuo, R. L. & Krug, R. M. (2006). The CPSF30 binding site on the NS1A protein of influenza A virus is a potential antiviral target. J Virol 80, 3957-3965.
32. Wang, S., Liu, Q., Pu, J., Li, Y., Keleta, L., Hu, Y. W., Liu, J. & Brown, E. G. (2008). Simplified recombinational approach for influenza A virus reverse genetics. J Virol Methods 151, 74-78.
33. Wang, W., Riedel, K., Lynch, P., Chien, C. Y., Montelione, G. T. & Krug, R. M. (1999). RNA binding by the novel helical domain of the influenza virus NS1 protein requires its dimer structure and a small number of specific basic amino acids. RNA 5, 195-205.
34. Ward, A. C. (1997). Virulence of influenza A virus for mouse lung. Virus Genes 14, 187-194.

1. An isolated NS1 variant polypeptide, protein or functional fragment thereof comprising a substitution in the amino acid sequence of the wild type NS1 protein at a position corresponding to Asp-2, Val-23, Leu-98, Phe-103, Ser-103, Met-106, Met-124, Asp-125, Val-180, Val-226 or Arg-227, or combinations thereof.

2. The isolated NS1 variant polypeptide of claim 1, wherein the amino acid sequence of the NS1 protein comprises a mutation selected from the group consisting of D2N (SEQ ID NO:1), V226I (SEQ ID NO:2), V23A (SEQ ID NO:3), F103L (SEQ ID NO:4), F103S (SEQ ID NO:5), M106I (SEQ ID NO:6), M106I+L98S (SEQ ID NO:7), M106V (SEQ ID NO:8), M106V+M124I (SEQ ID NO:9), D125G (SEQ ID NO:10), V180A (SEQ ID NO:11) and R227K (SEQ ID NO:12).

3. An isolated polynucleotide which encodes an NS1 variant polypeptide, protein or functional fragment thereof comprising a substitution in the amino acid sequence of the wild type NS1 protein at a position corresponding to Asp-2, Val-23, Leu-98, Phe-103, Met-106, Met-124, Asp-125, Val-180, Val-226 or Arg-227, or combinations thereof.

4. The isolated polynucleotide of claim 3, which encodes the amino acid sequence of the NS1 protein comprising a mutation selected from the group consisting of D2N (SEQ ID NO:1), V226I (SEQ ID NO:2), V23A (SEQ ID NO:3), F103L (SEQ ID NO:4), F103S (SEQ ID NO:5), M106I (SEQ ID NO:6), M106I+L98S (SEQ ID NO:7), M106V (SEQ ID NO:8), M106V+M124I (SEQ ID NO:9), D125G (SEQ ID NO:10), V180A (SEQ ID NO:11) and R227K (SEQ ID NO:12).

5. The isolated polynucleotide of claim 3, wherein said polynucleotide is a DNA, cDNA or RNA.

6. A vector comprising the isolated polynucleotide of claim 3.

7. The vector of claim 6, wherein the vector is a plasmid, a cosmid, a phage, a virus, or a fragment of a virus.

8. The vector of claim 6, wherein the vector is an expression vector.

9. A cell comprising the isolated polynucleotide of claim 3.

10. A cell comprising the vector of claim 6.

11. An NS1 mutant influenza virus comprising a coding sequence which encodes the polypeptide, protein or functional fragment thereof of claim 1.

12. An NS1 mutant influenza virus comprising a polynucleotide of claim 3.

13. A composition containing the NS1 mutant influenza virus of claim 11.

14. A composition containing the NS1 mutant influenza virus of claim 12.

15. A method to increase influenza protein and/or virus production comprising administering a composition of claim 13 to a cell, cell extract, tissue and/or tissue extract, and inducing protein and/or virus production in said cell, cell extract, tissue and/or tissue extract.

16. A method to increase influenza protein and/or virus production comprising administering a composition of claim 14 to a cell, cell extract, tissue and/or tissue extract, and inducing protein and/or virus production in said cell, cell extract, tissue and/or tissue extract.

17. The NS1 mutant influenza virus of claim 11, wherein the virus is a recombinant, reassortment, genetically engineered and/or attenuated influenza virus.

18. The NS1 mutant influenza virus of claim 12, wherein the virus is a recombinant, reassortment, genetically engineered and/or attenuated influenza virus.

19. An influenza viral master donor virus incorporating the coding sequence of a NS1 variant polypeptide, protein or functional fragment of claim 1.

20. The influenza viral master donor virus according to claim 19, wherein the coding sequence, when incorporated into the influenza viral master donor virus, causes the virus to exhibit an increased IFN inducing and high virus yield and/or protein synthesis phenotype.

21. A method of inducing an immune response comprising administering to a subject the NS1 mutant influenza virus of claim 11 in an amount sufficient to induce an immune response in said subject.

22. The method of claim 21, wherein the isolated NS1 variant polypeptide, protein or functional fragment is expressed by the NS1 mutant influenza virus.

23. A method of inducing an immune response comprising administering to a subject the NS1 mutant influenza virus of claim 12 in an amount sufficient to induce an immune response in said subject.

24. The method of claim 23, wherein the subject is an animal.

25. The method of claim 23, wherein the subject is a human.

26. The method of claim 21, wherein the subject is a animal.

27. The method of claim 23, wherein the NS1 mutant influenza virus is provided in a formulation for administration to the subject orally, intradermally, intramuscularly, intraperitoneally, intravenously, or subcutaneously.

28. An immunogenic composition containing an effective amount of the NS1 mutant influenza virus of claim 11.

29. The immunogenic composition of claim 28, wherein the NS1 mutant influenza virus is formulated for administration orally, intradermally, intramuscularly, intraperitoneally, intravenously, or subcutaneously.

30. A vaccine comprising the immunogenic composition of claim 28.

31. A method of producing an influenza vaccine comprising: (a) growing the NS1 mutant influenza virus of claim 11; (b) purifying the virus; and (c) combining the purified virus with a pharmaceutically acceptable excipient.

32. The method of claim 31, wherein the NS1 mutant influenza virus is grown in embryonated chicken eggs.

33. The method of claim 31, wherein the NS1 mutant influenza virus is inactivated prior to step (c).

34. A method of producing an influenza vaccine comprising: (a) preparing an NS1 variant polypeptide, protein or functional fragment thereof comprising a substitution in the amino acid sequence of the wild type NS1 protein at a position corresponding to Asp-2, Val-23, Leu-98, Phe-103, Ser-103, Met-106, Met-124, Asp-125, Val-180, Val-226 or Arg-227, or combinations thereof, or a polynucleotide encoding the NS1 variant polypeptide, protein or functional fragment thereof; (b) purifying the polypeptiptide, protein or functional fragment thereof or said polynucleotide; and (c) combining the NS1 variant polypeptide, protein or functional fragment thereof or said polynucleotide with an influenza antigen and a pharmaceutically acceptable carrier or excipient.

35. The method of claim 34, wherein the polypeptide or polynucleotide is prepared using a cell-based or synthetic method.


SEQUENCE LISTING (26 sequences)

SEQ ID NO: 1 - protein, 237 aa, Artificial Sequence
  NS1 D2N mutation
MNSNTVSSFQVDCFLWHVRKQVVDQELGDAPFLDRLRRDQKSLRGRGSTLGLNIEAATRVGKQIVERILKEESDEALKMTMASAPASRYLTDMTIEELSRDWFMLMPKQKVEGPLCIRIDQAIMDKNIMLKANFSVIFDRLETLILLRAFTEEGAIVGEISPLPSLPGHTIEDVKNAIGVLIGGLEWNDNTVRVSKTLQRFAWGSSNENGRPPLTPKQKRKMARTVRSKVRRDKMAD

SEQ ID NO: 2 - protein, 237 aa, Artificial Sequence
  NS1 V226I mutation
MDSNTVSSFQVDCFLWHVRKQVVDQELGDAPFLDRLRRDQKSLRGRGSTLGLNIEAATRVGKQIVERILKEESDEALKMTMASAPASRYLTDMTIEELSRDWFMLMPKQKVEGPLCIRIDQAIMDKNIMLKANFSVIFDRLETLILLRAFTEEGAIVGEISPLPSLPGHTIEDVKNAIGVLIGGLEWNDNTVRVSKTLQRFAWGSSNENGRPPLTPKQKRKMARTIRSKVRRDKMAD

SEQ ID NO: 3 - protein, 237 aa, Artificial Sequence
  NS1 V23A mutation
MDSNTVSSFQVDCFLWHVRKQVADQELGDAPFLDRLRRDQKSLRGRGSTLGLNIEAATRVGKQIVERILKEESDEALKMTMASAPASRYLTDMTIEELSRDWFMLMPKQKVEGPLCIRIDQAIMDKNIMLKANFSVIFDRLETLILLRAFTEEGAIVGEISPLPSLPGHTIEDVKNAIGVLIGGLEWNDNTVRVSKTLQRFAWGSSNENGRPPLTPKQKRKMARTVRSKVRRDKMAD

SEQ ID NO: 4 - protein, 237 aa, Artificial Sequence
  NS1 F103L mutation
MDSNTVSSFQVDCFLWHVRKQVVDQELGDAPFLDRLRRDQKSLRGRGSTLGLNIEAATRVGKQIVERILKEESDEALKMTMASAPASRYLTDMTIEELSRDWLMLMPKQKVEGPLCIRIDQAIMDKNIMLKANFSVIFDRLETLILLRAFTEEGAIVGEISPLPSLPGHTIEDVKNAIGVLIGGLEWNDNTVRVSKTLQRFAWGSSNENGRPPLTPKQKRKMARTVRSKVRRDKMAD

SEQ ID NO: 5 - protein, 237 aa, Artificial Sequence
  NS1 F103S mutation
MDSNTVSSFQVDCFLWHVRKQVVDQELGDAPFLDRLRRDQKSLRGRGSTLGLNIEAATRVGKQIVERILKEESDEALKMTMASAPASRYLTDMTIEELSRDWSMLMPKQKVEGPLCIRIDQAIMDKNIMLKANFSVIFDRLETLILLRAFTEEGAIVGEISPLPSLPGHTIEDVKNAIGVLIGGLEWNDNTVRVSKTLQRFAWGSSNENGRPPLTPKQKRKMARTVRSKVRRDKMAD

SEQ ID NO: 6 - protein, 237 aa, Artificial Sequence
  NS1 M106I mutation
MDSNTVSSFQVDCFLWHVRKQVVDQELGDAPFLDRLRRDQKSLRGRGSTLGLNIEAATRVGKQIVERILKEESDEALKMTMASAPASRYLTDMTIEELSRDWFMLIPKQKVEGPLCIRIDQAIMDKNIMLKANFSVIFDRLETLILLRAFTEEGAIVGEISPLPSLPGHTIEDVKNAIGVLIGGLEWNDNTVRVSKTLQRFAWGSSNENGRPPLTPKQKRKMARTVRSKVRRDKMAD

SEQ ID NO: 7 - protein, 237 aa, Artificial Sequence
  NS1 M106I + L98S mutation
MDSNTVSSFQVDCFLWHVRKQVVDQELGDAPFLDRLRRDQKSLRGRGSTLGLNIEAATRVGKQIVERILKEESDEALKMTMASAPASRYLTDMTIEESSRDWFMLIPKQKVEGPLCIRIDQAIMDKNIMLKANFSVIFDRLETLILLRAFTEEGAIVGEISPLPSLPGHTIEDVKNAIGVLIGGLEWNDNTVRVSKTLQRFAWGSSNENGRPPLTPKQKRKMARTVRSKVRRDKMAD

SEQ ID NO: 8 - protein, 237 aa, Artificial Sequence
  NS1 M106V mutation
MDSNTVSSFQVDCFLWHVRKQVVDQELGDAPFLDRLRRDQKSLRGRGSTLGLNIEAATRVGKQIVERILKEESDEALKMTMASAPASRYLTDMTIEELSRDWFMLVPKQKVEGPLCIRIDQAIMDKNIMLKANFSVIFDRLETLILLRAFTEEGAIVGEISPLPSLPGHTIEDVKNAIGVLIGGLEWNDNTVRVSKTLQRFAWGSSNENGRPPLTPKQKRKMARTVRSKVRRDKMAD

SEQ ID NO: 9 - protein, 237 aa, Artificial Sequence
  NS1 M106V-M124I mutation
MDSNTVSSFQVDCFLWHVRKQVVDQELGDAPFLDRLRRDQKSLRGRGSTLGLNIEAATRVGKQIVERILKEESDEALKMTMASAPASRYLTDMTIEELSRDWFMLVPKQKVEGPLCIRIDQAIIDKNIMLKANFSVIFDRLETLILLRAFTEEGAIVGEISPLPSLPGHTIEDVKNAIGVLIGGLEWNDNTVRVSKTLQRFAWGSSNENGRPPLTPKQKRKMARTVRSKVRRDKMAD

SEQ ID NO: 10 - protein, 237 aa, Artificial Sequence
  NS1 D125G mutation
MDSNTVSSFQVDCFLWHVRKQVVDQELGDAPFLDRLRRDQKSLRGRGSTLGLNIEAATRVGKQIVERILKEESDEALKMTMASAPASRYLTDMTIEELSRDWFMLMPKQKVEGPLCIRIDQAIMGKNIMLKANFSVIFDRLETLILLRAFTEEGAIVGEISPLPSLPGHTIEDVKNAIGVLIGGLEWNDNTVRVSKTLQRFAWGSSNENGRPPLTPKQKRKMARTVRSKVRRDKMAD

SEQ ID NO: 11 - protein, 237 aa, Artificial Sequence
  NS1 V180A mutation
MDSNTVSSFQVDCFLWHVRKQVVDQELGDAPFLDRLRRDQKSLRGRGSTLGLNIEAATRVGKQIVERILKEESDEALKMTMASAPASRYLTDMTIEELSRDWFMLMPKQKVEGPLCIRIDQAIMDKNIMLKANFSVIFDRLETLILLRAFTEEGAIVGEISPLPSLPGHTIEDVKNAIGALIGGLEWNDNTVRVSKTLQRFAWGSSNENGRPPLTPKQKRKMARTVRSKVRRDKMAD

SEQ ID NO: 12 - protein, 237 aa, Artificial Sequence
  NS1 R227K mutation
MDSNTVSSFQVDCFLWHVRKQVVDQELGDAPFLDRLRRDQKSLRGRGSTLGLNIEAATRVGKQIVERILKEESDEALKMTMASAPASRYLTDMTIEELSRDWFMLMPKQKVEGPLCIRIDQAIMDKNIMLKANFSVIFDRLETLILLRAFTEEGAIVGEISPLPSLPGHTIEDVKNAIGVLIGGLEWNDNTVRVSKTLQRFAWGSSNENGRPPLTPKQKRKMARTVKSKVRRDKMAD

SEQ ID NO: 13 - DNA, 855 bp, Artificial Sequence
  NS1 D2N mutation
atgaantctaacactgtgtcaagttttcaggtagattgcttcctttggcatgtccgaaaacaagttgtagaccaagaactaggtgatgccccattccttgatcggcttcgccgagatcagaagtccctaaggggaagaggcagcactctcggtctaaacatcgaagcagccacccgtgttggaaagcagatagtagagaggattctgaaggaagaatccgatgaggcacttaaaatgaccatggcctccgcacctgcttcgcgatacctaactgacatgactattgaggaattgtcaagggactggttcatgctaatgcccaagcagaaagtggaaggacctctttgcatcagaatagaccaggcaatcatggataagaacatcatgttgaaagcgaatttcagtgtgatttttgaccggctagagaccctaatattactaagggctttcaccgaagagggagcaattgttggcgaaatctcaccattgccttctcttccaggacatactattgaggatgtcaaaaatgcaattggggtcctcatcggaggacttgaatggaatgataacacagttcgagtctctaaaactctacagagattcgcttggggaagcagtaatgagaatgggagacctccactcactccaaaacagaaacggaaaatggcgagaacagttaggtcaaaagttcgaagagataagatggctgattgaagaagtgagacacagattgaagacaacagagaatagttttgagcaaataacatttatgcaagccttacagctactatttgaagtggaacaggagataagaactttctcgtttcagcttatttaatgataaaaaacaccctt

SEQ ID NO: 14 - DNA, 855 bp, Artificial Sequence
  NS1 V226I mutation
atggattctaacactgtgtcaagttttcaggtagattgcttcctttggcatgtccgaaaacaagttgtagaccaagaactaggtgatgccccattccttgatcggcttcgccgagatcagaagtccctaaggggaagaggcagcactctcggtctaaacatcgaagcagccacccgtgttggaaagcagatagtagagaggattctgaaggaagaatccgatgaggcacttaaaatgaccatggcctccgcacctgcttcgcgatacctaactgacatgactattgaggaattgtcaagggactggttcatgctaatgcccaagcagaaagtggaaggacctctttgcatcagaatagaccaggcaatcatggataagaacatcatgttgaaagcgaatttcagtgtgatttttgaccggctagagaccctaatattactaagggctttcaccgaagagggagcaattgttggcgaaatctcaccattgccttctcttccaggacatactattgaggatgtcaaaaatgcaattggggtcctcatcggaggacttgaatggaatgataacacagttcgagtctctaaaactctacagagattcgcttggggaagcagtaatgagaatgggagacctccactcactccaaaacagaaacggaaaatggcgagaacaatnaggtcaaaagttcgaagagataagatggctgattgaagaagtgagacacagattgaagacaacagagaatagttttgagcaaataacatttatgcaagccttacagctactatttgaagtggaacaggagataagaactttctcgtttcagcttatttaatgataaaaaacaccctt

SEQ ID NO: 15 - DNA, 855 bp, Artificial Sequence
  NS1 V23A mutation
atggattctaacactgtgtcaagttttcaggtagattgcttcctttggcatgtccgaaaacaagttgcngaccaagaactaggtgatgccccattccttgatcggcttcgccgagatcagaagtccctaaggggaagaggcagcactctcggtctaaacatcgaagcagccacccgtgttggaaagcagatagtagagaggattctgaaggaagaatccgatgaggcacttaaaatgaccatggcctccgcacctgcttcgcgatacctaactgacatgactattgaggaattgtcaagggactggttcatgctaatgcccaagcagaaagtggaaggacctctttgcatcagaatagaccaggcaatcatggataagaacatcatgttgaaagcgaatttcagtgtgatttttgaccggctagagaccctaatattactaagggctttcaccgaagagggagcaattgttggcgaaatctcaccattgccttctcttccaggacatactattgaggatgtcaaaaatgcaattggggtcctcatcggaggacttgaatggaatgataacacagttcgagtctctaaaactctacagagattcgcttggggaagcagtaatgagaatgggagacctccactcactccaaaacagaaacggaaaatggcgagaacagttaggtcaaaagttcgaagagataagatggctgattgaagaagtgagacacagattgaagacaacagagaatagttttgagcaaataacatttatgcaagccttacagctactatttgaagtggaacaggagataagaactttctcgtttcagcttatttaatgataaaaaacaccctt

SEQ ID NO: 16 - DNA, 855 bp, Artificial Sequence
  NS1 F103L mutation
atggattctaacactgtgtcaagttttcaggtagattgcttcctttggcatgtccgaaaacaagttgtagaccaagaactaggtgatgccccattccttgatcggcttcgccgagatcagaagtccctaaggggaagaggcagcactctcggtctaaacatcgaagcagccacccgtgttggaaagcagatagtagagaggattctgaaggaagaatccgatgaggcacttaaaatgaccatggcctccgcacctgcttcgcgatacctaactgacatgactattgaggaattgtcaagggactggnnnatgctaatgcccaagcagaaagtggaaggacctctttgcatcagaatagaccaggcaatcatggataagaacatcatgttgaaagcgaatttcagtgtgatttttgaccggctagagaccctaatattactaagggctttcaccgaagagggagcaattgttggcgaaatctcaccattgccttctcttccaggacatactattgaggatgtcaaaaatgcaattggggtcctcatcggaggacttgaatggaatgataacacagttcgagtctctaaaactctacagagattcgcttggggaagcagtaatgagaatgggagacctccactcactccaaaacagaaacggaaaatggcgagaacagttaggtcaaaagttcgaagagataagatggctgattgaagaagtgagacacagattgaagacaacagagaatagttttgagcaaataacatttatgcaagccttacagctactatttgaagtggaacaggagataagaactttctcgtttcagcttatttaatgataaaaaacaccctt

SEQ ID NO: 17 - DNA, 855 bp, Artificial Sequence; identical to SEQ ID NO: 16
  NS1 F103S mutation

SEQ ID NO: 18 - DNA, 855 bp, Artificial Sequence
  NS1 M106I mutation
atggattctaacactgtgtcaagttttcaggtagattgcttcctttggcatgtccgaaaacaagttgtagaccaagaactaggtgatgccccattccttgatcggcttcgccgagatcagaagtccctaaggggaagaggcagcactctcggtctaaacatcgaagcagccacccgtgttggaaagcagatagtagagaggattctgaaggaagaatccgatgaggcacttaaaatgaccatggcctccgcacctgcttcgcgatacctaactgacatgactattgaggaattgtcaagggactggttcatgctaatncccaagcagaaagtggaaggacctctttgcatcagaatagaccaggcaatcatggataagaacatcatgttgaaagcgaatttcagtgtgatttttgaccggctagagaccctaatattactaagggctttcaccgaagagggagcaattgttggcgaaatctcaccattgccttctcttccaggacatactattgaggatgtcaaaaatgcaattggggtcctcatcggaggacttgaatggaatgataacacagttcgagtctctaaaactctacagagattcgcttggggaagcagtaatgagaatgggagacctccactcactccaaaacagaaacggaaaatggcgagaacagttaggtcaaaagttcgaagagataagatggctgattgaagaagtgagacacagattgaagacaacagagaatagttttgagcaaataacatttatgcaagccttacagctactatttgaagtggaacaggagataagaactttctcgtttcagcttatttaatgataaaaaacaccctt

SEQ ID NO: 19 - DNA, 855 bp, Artificial Sequence
  NS1 M106I + L98S mutation
atggattctaacactgtgtcaagttttcaggtagattgcttcctttggcatgtccgaaaacaagttgtagaccaagaactaggtgatgccccattccttgatcggcttcgccgagatcagaagtccctaaggggaagaggcagcactctcggtctaaacatcgaagcagccacccgtgttggaaagcagatagtagagaggattctgaaggaagaatccgatgaggcacttaaaatgaccatggcctccgcacctgcttcgcgatacctaactgacatgactattgaggaannntcaagggactggttcatgctaatncccaagcagaaagtggaaggacctctttgcatcagaatagaccaggcaatcatggataagaacatcatgttgaaagcgaatttcagtgtgatttttgaccggctagagaccctaatattactaagggctttcaccgaagagggagcaattgttggcgaaatctcaccattgccttctcttccaggacatactattgaggatgtcaaaaatgcaattggggtcctcatcggaggacttgaatggaatgataacacagttcgagtctctaaaactctacagagattcgcttggggaagcagtaatgagaatgggagacctccactcactccaaaacagaaacggaaaatggcgagaacagttaggtcaaaagttcgaagagataagatggctgattgaagaagtgagacacagattgaagacaacagagaatagttttgagcaaataacatttatgcaagccttacagctactatttgaagtggaacaggagataagaactttctcgtttcagcttatttaatgataaaaaacaccctt

SEQ ID NO: 20 - DNA, 855 bp, Artificial Sequence
  NS1 M106V mutation
atggattctaacactgtgtcaagttttcaggtagattgcttcctttggcatgtccgaaaacaagttgtagaccaagaactaggtgatgccccattccttgatcggcttcgccgagatcagaagtccctaaggggaagaggcagcactctcggtctaaacatcgaagcagccacccgtgttggaaagcagatagtagagaggattctgaaggaagaatccgatgaggcacttaaaatgaccatggcctccgcacctgcttcgcgatacctaactgacatgactattgaggaattgtcaagggactggttcatgctagtncccaagcagaaagtggaaggacctctttgcatcagaatagaccaggcaatcatggataagaacatcatgttgaaagcgaatttcagtgtgatttttgaccggctagagaccctaatattactaagggctttcaccgaagagggagcaattgttggcgaaatctcaccattgccttctcttccaggacatactattgaggatgtcaaaaatgcaattggggtcctcatcggaggacttgaatggaatgataacacagttcgagtctctaaaactctacagagattcgcttggggaagcagtaatgagaatgggagacctccactcactccaaaacagaaacggaaaatggcgagaacagttaggtcaaaagttcgaagagataagatggctgattgaagaagtgagacacagattgaagacaacagagaatagttttgagcaaataacatttatgcaagccttacagctactatttgaagtggaacaggagataagaactttctcgtttcagcttatttaatgataaaaaacaccctt

SEQ ID NO: 21 - DNA, 855 bp, Artificial Sequence
  NS1 M106V + M124I mutation
atggattctaacactgtgtcaagttttcaggtagattgcttcctttggcatgtccgaaaacaagttgtagaccaagaactaggtgatgccccattccttgatcggcttcgccgagatcagaagtccctaaggggaagaggcagcactctcggtctaaacatcgaagcagccacccgtgttggaaagcagatagtagagaggattctgaaggaagaatccgatgaggcacttaaaatgaccatggcctccgcacctgcttcgcgatacctaactgacatgactattgaggaattgtcaagggactggttcatgctagtncccaagcagaaagtggaaggacctctttgcatcagaatagaccaggcaatcatngataagaacatcatgttgaaagcgaatttcagtgtgatttttgaccggctagagaccctaatattactaagggctttcaccgaagagggagcaattgttggcgaaatctcaccattgccttctcttccaggacatactattgaggatgtcaaaaatgcaattggggtcctcatcggaggacttgaatggaatgataacacagttcgagtctctaaaactctacagagattcgcttggggaagcagtaatgagaatgggagacctccactcactccaaaacagaaacggaaaatggcgagaacagttaggtcaaaagttcgaagagataagatggctgattgaagaagtgagacacagattgaagacaacagagaatagttttgagcaaataacatttatgcaagccttacagctactatttgaagtggaacaggagataagaactttctcgtttcagcttatttaatgataaaaaacaccctt

SEQ ID NO: 22 - DNA, 855 bp, Artificial Sequence
  NS1 D125G mutation
atggattctaacactgtgtcaagttttcaggtagattgcttcctttggcatgtccgaaaacaagttgtagaccaagaactaggtgatgccccattccttgatcggcttcgccgagatcagaagtccctaaggggaagaggcagcactctcggtctaaacatcgaagcagccacccgtgttggaaagcagatagtagagaggattctgaaggaagaatccgatgaggcacttaaaatgaccatggcctccgcacctgcttcgcgatacctaactgacatgactattgaggaattgtcaagggactggttcatgctaatgcccaagcagaaagtggaaggacctctttgcatcagaatagaccaggcaatcatgggnaagaacatcatgttgaaagcgaatttcagtgtgatttttgaccggctagagaccctaatattactaagggctttcaccgaagagggagcaattgttggcgaaatctcaccattgccttctcttccaggacatactattgaggatgtcaaaaatgcaattggggtcctcatcggaggacttgaatggaatgataacacagttcgagtctctaaaactctacagagattcgcttggggaagcagtaatgagaatgggagacctccactcactccaaaacagaaacggaaaatggcgagaacagttaggtcaaaagttcgaagagataagatggctgattgaagaagtgagacacagattgaagacaacagagaatagttttgagcaaataacatttatgcaagccttacagctactatttgaagtggaacaggagataagaactttctcgtttcagcttatttaatgataaaaaacaccctt

SEQ ID NO: 23 - DNA, 855 bp, Artificial Sequence
  NS1 V180A mutation
atggattctaacactgtgtcaagttttcaggtagattgcttcctttggcatgtccgaaaacaagttgtagaccaagaactaggtgatgccccattccttgatcggcttcgccgagatcagaagtccctaaggggaagaggcagcactctcggtctaaacatcgaagcagccacccgtgttggaaagcagatagtagagaggattctgaaggaagaatccgatgaggcacttaaaatgaccatggcctccgcacctgcttcgcgatacctaactgacatgactattgaggaattgtcaagggactggttcatgctaatgcccaagcagaaagtggaaggacctctttgcatcagaatagaccaggcaatcatggataagaacatcatgttgaaagcgaatttcagtgtgatttttgaccggctagagaccctaatattactaagggctttcaccgaagagggagcaattgttggcgaaatctcaccattgccttctcttccaggacatactattgaggatgtcaaaaatgcaattggggcnctcatcggaggacttgaatggaatgataacacagttcgagtctctaaaactctacagagattcgcttggggaagcagtaatgagaatgggagacctccactcactccaaaacagaaacggaaaatggcgagaacagttaggtcaaaagttcgaagagataagatggctgattgaagaagtgagacacagattgaagacaacagagaatagttttgagcaaataacatttatgcaagccttacagctactatttgaagtggaacaggagataagaactttctcgtttcagcttatttaatgataaaaaacaccctt

SEQ ID NO: 24 - DNA, 855 bp, Artificial Sequence
  NS1 R227K mutation
atggattctaacactgtgtcaagttttcaggtagattgcttcctttggcatgtccgaaaacaagttgtagaccaagaactaggtgatgccccattccttgatcggcttcgccgagatcagaagtccctaaggggaagaggcagcactctcggtctaaacatcgaagcagccacccgtgttggaaagcagatagtagagaggattctgaaggaagaatccgatgaggcacttaaaatgaccatggcctccgcacctgcttcgcgatacctaactgacatgactattgaggaattgtcaagggactggttcatgctaatgcccaagcagaaagtggaaggacctctttgcatcagaatagaccaggcaatcatggataagaacatcatgttgaaagcgaatttcagtgtgatttttgaccggctagagaccctaatattactaagggctttcaccgaagagggagcaattgttggcgaaatctcaccattgccttctcttccaggacatactattgaggatgtcaaaaatgcaattggggtcctcatcggaggacttgaatggaatgataacacagttcgagtctctaaaactctacagagattcgcttggggaagcagtaatgagaatgggagacctccactcactccaaaacagaaacggaaaatggcgagaacagttaantcaaaagttcgaagagataagatggctgattgaagaagtgagacacagattgaagacaacagagaatagttttgagcaaataacatttatgcaagccttacagctactatttgaagtggaacaggagataagaactttctcgtttcagcttatttaatgataaaaaacaccctt

SEQ ID NO: 25 - protein, 237 aa, Influenza A virus
MDSNTVSSFQVDCFLWHVRKQVVDQELGDAPFLDRLRRDQKSLRGRGSTLGLNIEAATRVGKQIVERILKEESDEALKMTMASAPASRYLTDMTIEELSRDWFMLMPKQKVEGPLCIRIDQAIMDKNIMLKANFSVIFDRLETLILLRAFTEEGAIVGEISPLPSLPGHTIEDVKNAIGVLIGGLEWNDNTVRVSKTLQRFAWGSSNENGRPPLTPKQKRKMARTVRSKVRRDKMAD

SEQ ID NO: 26 - DNA, 855 bp, Influenza A virus
atggattctaacactgtgtcaagttttcaggtagattgcttcctttggcatgtccgaaaacaagttgtagaccaagaactaggtgatgccccattccttgatcggcttcgccgagatcagaagtccctaaggggaagaggcagcactctcggtctaaacatcgaagcagccacccgtgttggaaagcagatagtagagaggattctgaaggaagaatccgatgaggcacttaaaatgaccatggcctccgcacctgcttcgcgatacctaactgacatgactattgaggaattgtcaagggactggttcatgctaatgcccaagcagaaagtggaaggacctctttgcatcagaatagaccaggcaatcatggataagaacatcatgttgaaagcgaatttcagtgtgatttttgaccggctagagaccctaatattactaagggctttcaccgaagagggagcaattgttggcgaaatctcaccattgccttctcttccaggacatactattgaggatgtcaaaaatgcaattggggtcctcatcggaggacttgaatggaatgataacacagttcgagtctctaaaactctacagagattcgcttggggaagcagtaatgagaatgggagacctccactcactccaaaacagaaacggaaaatggcgagaacagttaggtcaaaagttcgaagagataagatggctgattgaagaagtgagacacagattgaagacaacagagaatagttttgagcaaataacatttatgcaagccttacagctactatttgaagtggaacaggagataagaactttctcgtttcagcttatttaatgataaaaaacaccctt